(19) United States
(12) Patent Application Publication
(10) Pub. No.: US 2024/0299497 A1
(43) Pub. Date: Sep. 12, 2024

(54) IMMUNOMODULATION FOR THE LONG TERM PREVENTION AND TREATMENT OF AUTOIMMUNE DISEASES AND FOREIGN TISSUE REJECTION

(71) Applicants: University of Louisville Research Foundation, Inc., Louisville, KY (US); The Curators of the University of Missouri, Columbia, MO (US)

(72) Inventor: Haval Shirwan, Columbia, MO (US)

(73) Assignees: University of Louisville Research Foundation, Inc., Louisville, KY (US); The Curators of the University of Missouri, Columbia, MO (US)

(21) Appl. No.: 18/673,037

(22) Filed: May 23, 2024

Related U.S. Application Data
(62) Division of application No. 15/736,088, filed on Dec. 13, 2017, now Pat. No. 12,023,367.
(60) Provisional application No. 62/181,815, filed on Jun. 19, 2015.

(51) Int. Cl. A61K 38/20; A61K 31/35; A61K 31/675; A61K 35/12; A61K 35/26; A61K 35/39; A61K 38/17; A61K 39/00; A61K 39/385; A61K 45/06; A61K 47/69; A61P 37/06; A61P 43/00

(52) U.S. Cl. CPC A61K 38/2013; A61K 35/12; A61K 35/26; A61K 35/39; A61K 38/177; A61K 39/0008; A61K 39/385; A61K 45/06; A61K 47/6901; A61P 37/06; A61P 43/00; A61K 31/35; A61K 31/675; A61K 2039/577; A61K 2039/58

(57) ABSTRACT

Described herein are pharmaceutical compositions, medicaments and methods for providing effective immunomodulation with chimeric proteins including FasL moieties for selective and long-lasting regulation of the immune response.

### CROSS REFERENCE TO RELATED APPLICATIONS

This application is a division of U.S. patent application Ser. No. 15/736,088, filed Dec. 13, 2017, which is the U.S. National Stage of International Application PCT/US2016/038185, filed Jun. 17, 2016, which claims priority to U.S. Provisional Patent Application No. 62/181,815, filed Jun. 19, 2015, each of which is incorporated herein by reference in its entirety.

### GOVERNMENT GRANT SUPPORT CLAUSE

This invention was made with government support under AI055456 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on May 10, 2024, is named 089885-0171_SL.xml and is 24,584 bytes in size.

### TECHNICAL FIELD

Described herein are pharmaceutical compositions and methods for modulating the immune system, such as for the prevention and treatment of autoimmune diseases and foreign graft rejection.

### BACKGROUND

Autoimmune diseases and graft rejection are well recognized problems. Current clinical practice is to administer immunosuppressants that prevent T-cell activity. Such immunosuppressants are administered for an extended period in autoimmune disease, and often for the lifetime of the patient who has received foreign grafts. The requirement for long term use of immunosuppressants makes successful treatment dependent on frequent medical monitoring, and exposes the patient to serious side effects from the drugs. As an alternative, it would be advantageous to develop a treatment that “teaches” the immune system to control/eliminate destructive immune responses generated by specific antigens.

The present inventor has previously demonstrated immunomodulation by display of SA-FasL on the surface of biological membranes modified with biotin. Injecting subjects with foreign cells decorated with SA-FasL has shown efficacy in modulating immune responses and achieving long term survival of tolerance to grafted tissues. Nevertheless, there remains a need for methods that “teach” the immune system to tolerate specific foreign and self antigens, that exhibit improved efficacy and are useful in a broad variety of applications.

### SUMMARY

The invention described herein relates to the surprising discovery that soluble SA-FasL, optionally together with IL-2, optionally together with FasL-decorated cells and/or IL-2-decorated cells, is useful in methods for inducing long-term and specific immunosuppression. Thus, described herein are compositions, medicaments and methods useful for immunomodulation, such as for long-term prevention and/or treatment of autoimmune diseases and long-term prevention and/or treatment of graft rejection.

In accordance with some embodiments, there are provided medicaments for use in inducing immune tolerance in a subject in need thereof, comprising (i) a chimeric FasL protein comprising a FasL moiety and a streptavidin or avidin moiety and (ii) an IL-2 protein. In accordance with any embodiments, the chimeric FasL protein and IL-2 protein may be provided in separate compositions or in the same composition.

The chimeric FasL protein may be selected from soluble chimeric FasL proteins comprising a FasL moiety and a streptavidin or avidin moiety, and chimeric FasL-decorated cells comprising a chimeric FasL protein comprising a FasL moiety and a streptavidin or avidin moiety bound to a cell surface via a biotin moiety on the cell surface. One embodiment of a chimeric FasL protein has the amino acid sequence of SEQ ID NO: 3.

The IL-2 protein may be selected from soluble IL-2 proteins, soluble chimeric IL-2 proteins comprising an IL-2 moiety and a streptavidin or avidin moiety, and chimeric IL-2-decorated cells comprising a chimeric IL-2 protein comprising an IL-2 moiety and a streptavidin or avidin moiety bound to a cell surface via a biotin moiety on the cell surface. One embodiment of a chimeric IL-2 protein has the amino acid sequence of SEQ ID NO: 9.

In one embodiment, the medicament comprises soluble chimeric FasL protein and soluble IL-2 protein or soluble chimeric IL-2 protein. In another embodiment, the medicament comprises chimeric FasL-decorated cells and soluble IL-2 protein or soluble chimeric IL-2 protein. In another, the medicament comprises soluble chimeric FasL protein and chimeric IL-2-decorated cells. In another, the medicament comprises chimeric FasL-decorated cells and chimeric IL-2-decorated cells. In specific embodiments, a medicament for use in inducing immune tolerance may comprise soluble chimeric FasL protein comprising a FasL moiety and a streptavidin or avidin moiety.

The medicaments are effective to induce immunomodulation in subjects in need of such immunomodulation. For example, a subject may be in in need of treatment for type 1 diabetes and the decorated cells, if present, may be islet cells, splenocytes, PBMC, bone marrow cells, mesenchymal stem cells, hematopoietic stem cells, stem cells, or induced pluripotent stem cells. Alternatively, the subject may be in need of the treatment or prevention of allograft rejection and the decorated cells, if present, may be cells from the allograft donor, such as cells selected from the group consisting of allograft bone marrow cells, allograft cardiac myocytes and allograft vascular cells, or other cells from the allograft donor.

The invention also includes methods for inducing immunomodulation. In accordance with some embodiments, there are provided methods of inducing immune tolerance in a subject in need thereof comprising administering to the individual (i) a chimeric FasL protein comprising a FasL moiety and a streptavidin or avidin moiety and (ii) an IL-2 protein. In accordance with any embodiments, the chimeric FasL protein and IL-2 protein may be administered together, or separately, in any order.

The chimeric FasL protein may be selected from soluble chimeric FasL proteins comprising a FasL moiety and a streptavidin or avidin moiety and chimeric FasL-decorated cells comprising a chimeric FasL protein comprising a FasL moiety and a streptavidin or avidin moiety bound to a cell surface via a biotin moiety on the cell surface. One embodiment of a chimeric FasL protein has the amino acid sequence of SEQ ID NO: 3.

The IL-2 protein may be selected from the group consisting of soluble IL-2 proteins, soluble chimeric IL-2 proteins comprising an IL-2 moiety and a streptavidin or avidin moiety; and chimeric IL-2-decorated cells comprising a chimeric IL-2 protein comprising an IL-2 moiety and a streptavidin or avidin moiety bound to a cell surface via a biotin moiety on the cell surface. One embodiment of a chimeric IL-2 protein has the amino acid sequence of SEQ ID NO: 9.

The methods may be used to induce immunomodulation in subjects in need of such immunomodulation. For example, a subject may be in need of treatment for type 1 diabetes and the decorated cells, if present, may be islet cells, PBMCs or splenocytes, bone marrow cells, mesenchymal stem cells, hematopoietic stem cells, stem cells, or induced pluripotent stem cells. In some embodiments, such a method may comprise administering to the subject in need thereof:
(a) chimeric FasL-decorated pancreatic islet cells;
(b)(i) soluble chimeric FasL protein and (ii) soluble IL-2 protein or soluble chimeric IL-2 protein, in amounts effective to induce immune tolerance to pancreatic islet cells; and
(c) optionally, administering an immunosuppressant, such as rapamycin or cyclophosamide.

In further embodiments, a subject is in need of the treatment or prevention of allograft rejection and the decorated cells, if present, are cells of the allograft, such as cells selected from the group consisting of allograft bone marrow cells, PBMC, mesenchymal stem cells, hematopoietic stem cells, stem cells, induced pluripotent stem cells, cardiac myocytes and vascular cells. In some embodiments, such a method of treating or preventing allograft rejection in a subject in need thereof, comprises (a) chimeric FasL-decorated cells from the allograft donor, such as cells selected from the group consisting of allograft bone marrow cells, allograft cardiac myocytes and allograft vascular cells, or other cells from the allograft donor; (b) administering (i) soluble chimeric FasL protein and (ii) soluble IL-2 protein or soluble chimeric IL-2 protein, in amounts effective to induce immune tolerance to the allograft cells; and optionally, administering an immunosuppressant, such as rapamycin or cyclophosamide. The cells from the allograft donor may also be decorated with one or both of FasL and IL-2.

In accordance with further embodiments, there are provided methods of treating or preventing autoimmunity in a subject in need thereof, comprising (a) administering to the subject cells decorated with chimeric FasL and optionally decorated with chimeric IL-2, and (b) administering (i) soluble chimeric FasL protein and (ii) soluble IL-2 protein or soluble chimeric IL-2 protein, in amounts effective to induce immune tolerance to the autoantigen; wherein the cells are selected from (i) a cell expressing an autoantigen (ii) a cell decorated with an autoantigen and (iii) a dendritic cell pulsed with the autoantigen. In particular embodiments, the cell expressing an autoantigen or the cell decorated with the autoantigen is obtained from the subject. In specific embodiments, the cell is selected from bone marrow cells, dendritic cells, PBMC, hematopoietic stem cells, and mesenchymal stem cells, including any of such cells obtained from the subject.

In accordance with further embodiments, there are provided methods of treating or preventing autoimmunity in a subject in need thereof, comprising (a) administering an autoantigen presented on a cell selected from the group consisting of (i) a cell expressing the autoantigen (ii) a cell decorated with the autoantigen and (iii) a dendritic cell pulsed with the autoantigen, wherein the cell optionally is decorated with chimeric FasL and/or chimeric IL-2, and (b) administering (i) soluble chimeric FasL protein and (ii) soluble IL-2 protein or soluble chimeric IL-2 protein, in amounts effective to induce immune tolerance to the autoantigen. In particular embodiments, the cell is obtained from the subject. In specific embodiments, the cell is selected from bone marrow cells, dendritic cells, PBMC, hematopoietic stem cells, and mesenchymal stem cells, including any of such cells obtained from the subject.

### BRIEF DESCRIPTION OF THE DRAWINGS

FIG. 1. Systemic immunomodulation with soluble SA-FasL synergizes with SA-FasL-engineered islets for long-term survival. BALB/c islets were engineered with SA-FasL (mSA-FasL) or SA control (mSA) proteins and transplanted under the kidney capsule of STZ diabetic C57BL/6 mice. Soluble (s)SA-FasL was administered i.p. at 2-5 μg/mouse on days 1, 3, 5, 7 post-transplantation. In selected groups rapamycin was used at 0.2 mg/kg daily for 15 days starting the day of transplantation.

FIG. 2. Survival of allogeneic islets transplanted intraportally with different treatment protocols. BALB/c islets were engineered with SA-FasL protein and transplanted intraportally into STZ diabetic C57BL/6 mice. Rapa was used daily at 0.2 mg/kg for 15 doses starting on day of transplantation. Soluble (s)FasL was used at the indicated dose and regimens.

FIG. 3. SA-FasL induces apoptosis in nonhuman primate (NHP) Teff cells. NHP (rhesus monkey) PBMCs were cultured with SA-FasL and apoptosis was assessed using Annexin V by gating on CD3⁺ T cells. Cells incubated with SA served as controls. Data is representative of PBMCs from 3 NHPs.

FIG. 4A-B. SA-FasL-engineered islet grafts overcome rejection in recipients with established donor-specific immune memory. C57BL/6 mice were transplanted with BALB/c skin grafts on day 0, followed by STZ treatment and transplantation of BALB/c SA-FasL engineered islet grafts under the kidney capsule on day 40-50 post-skin transplantation. Mice transplanted with SA protein engineered islets (SA-islet) served as control. Rapamycin was given i.p. daily at 0.2 mg/kg for 15 doses starting on the day of islet transplantation. Graft survival is shown in FIG. 4A. The kidney bearing the transplanted islet graft was surgically removed for 3 mice in the SA-FasL group on day 109 post islet transplantation to confirm graft-dependent euglycemia (FIG. 4B). Animals were sacrificed 3 days post-nephrectomy after the confirmation of hyperglycemia.

FIG. 5. Systemic treatment with SA-FasL protein delays the onset and incidence of Type 1 Diabetes in NOD. Female NOD mice were treated with the indicated agents at 10-week of age. Mice were then monitored for hyperglycemia by urine glucose test, which was confirmed by a blood glucose test. Mice with >200 mg/dl blood glucose levels were considered diabetic. Soluble (s)SA-FasL in PBS was used at 500 ng/injection intraperitoneally daily for 30 days. IL-2 was used at 3000 IU/injection daily for 30 days, whereas rapamycin was used daily at 0.2 mg/kg for 30 days. Female mice without any treatment were used as controls. In the SA-FasL only treatment group, 3 diabetic mice were treated with an additional 10 daily doses (black arrows) of SA-FasL soon after incidence of diabetes, resulting in temporary remission of hyperglycemia in ⅔ of those mice.

FIG. 6A-B. Combined use of SA-FasL and IL-2 prevents allogeneic islet graft rejection. BALB/c islets were transplanted intraportally into STZ diabetic age-matched males and females C57BL/6 mice. Recipients were treated with the indicated regimens of SA-FasL and IL-2 and monitored for graft rejection. In selected groups islets were either engineered with SA-FasL (SA-FasL-islet) or both SA-FasL and SA-IL2 (SA-FasL/SA-IL2-islet. Mice injected with soluble SA served as controls. Control islet and islet+IL-2 groups are the same for both panels for comparison purposes.

FIG. 7. Synergy between SA-FasL and IL-2 in inducing tolerance to cardiac allografts in the BALB/c-to-C57BL/6 model. C57BL/6 recipients of allogeneic BALB/c hearts were treated i.p. with the indicated doses of IL-2 and SA-FasL and mice were monitored for graft survival.

FIG. 8. Increased CD4⁺CD25⁺FoxP3⁺ and CD8⁺CD122⁺PD1⁺ T reg cells within the graft. Frequencies of CD4⁺CD25⁺FoxP3⁺ and CD8⁺CD122⁺PD1⁺ T reg cells within heart grafts at rejection or experimental end-point (LT; >100 days) shown as percentage of total CD4⁺ (A) and CD8⁺ T (B) cells.

FIG. 9. CD4⁺CD25⁺FoxP3⁺ T reg cells are important to tolerance induced by SA-FasL+IL-2. C57BL/6.FoxP3^{DTR} recipients of allogeneic BALB/c hearts were treated with IL-2 and SA-FasL regimen used in FIG. 7 to induce tolerance. Mice were injected i.p. with two doses of 50 ng/kg diphtheria toxin (DT) on two consecutive days to deplete T reg cells expressing DT receptor under the control of FoxP3. T reg depletion was confirmed by flow cytometry and mice were monitored for graft survival.

FIG. 10. CD8⁺CD122⁺PD1⁺ T cells have regulatory function. CD8⁺CD122⁺PD1⁺ and CD8⁺CD122⁺PD1⁻ cells were flow sorted from long-term heart grafts infiltrating lymphocytes and 5×10³ cells from each population was co-transferred with 1×10⁶ flow sorted naïve spleen CD4⁺CD25⁻ Teff cells into Rag^{-/-} mice. One day later, these animals were transplanted with BALB/c hearts and graft rejection was monitored. Animals receiving flow sorted naïve Teff cells and naïve CD4⁺CD25⁺FoxP3⁺ (GFP) cells served as controls.

FIG. 11A-C. SA-FasL and IL-2 combination as an effective means of establishing mixed hematopoietic allogeneic chimerism. C57BL/6 mice were subjected to 300 cGy total body irradiation followed by intravenous transplantation of 30'10⁶ unmodified (BMC) or SA-FasL (mFasL-BMC) or the combination of SA-FasL and SA-IL-2 (mFasL/IL-2-BMC) one day later. Selected groups of mice were also treated i.p. with 500 ng/daily for 15 doses of soluble SA-FasL (sFasL), 25,000 IU/daily for 7 doses of soluble IL-2 (sIL-2; human IL-2 obtained commercially or produced in house using insect cells transfected with human IL-2 cDNA engineered to include a 6×His tag (SEQ ID NO:11) for purification) or combination of both molecules (sFasL+sIL-2). The indicated groups also were treated i.p. with 10 daily doses of rapamycin at 0.5 mg/kg. Percent donor chimerism at various times post-transplantation was assessed using peripheral blood lymphocytes with antibodies against donor class I MHC molecule in flow cytometry. (FIG. 11A) average donor chimerism, (FIG. 11B) percent engraftment, (FIG. 11C) Long-term chimeric mice reject C3H third-party, but not BALB/c donor skin, demonstrating donor-specific tolerance.

FIG. 12A-C. Cyclophosphamide works in synergy with SA-FasL and IL-2 combination in establishing mixed hematopoietic allogeneic chimerism. C57BL/6 mice were subjected to 100 cGy total body irradiation followed by intravenous transplantation of 30×10⁶ unmodified (BMC) or the combination of SA-FasL and SA-IL-2 (mFasL/IL-2-BMC) one day later. Selected groups of mice were also treated i.p. with cyclophosphamide (200 mg/kg) on day+2. All groups were treated i.p. with rapamycin (2 mg/kg) daily for 10 doses starting on day 6 pot-transplantation. The test group was also treated starting on day 6 post-transplantation with both soluble SA-FasL (sFasL; 500 ng/daily for 15 doses) and IL-2 (25,000 IU/daily for 7 doses). Percent donor chimerism at various times post-transplantation was assessed using peripheral blood lymphocytes with antibodies against donor class I MHC molecule in flow cytometry. (FIG. 12A) average donor chimerism, (FIG. 12B) percent engraftment, (FIG. 12C) long-term chimeric mice reject C3H third-party, but not BALB/c donor skin, demonstrating donor-specific tolerance.

FIG. 13A-E. Mice develop long-term multilineage donor chimerism. Various tissues were harvested from mice with long-term donor mixed chimerism that was established through complete treatment with mFasL/IL-2-BMC+CYP (d+2)+sFasL+sIL-2+Rapa, as in FIG. 12. The tissues were, processed into single cell suspension, stained with antibodies to the indicated cell surface markers, and analyzed using flow cytometry. FIG. 13A shows total percentage of donor cells in the indicated tissues; FIG. 13B, donor cell type as a percentage of total donor cells in peripheral blood lymphocytes; FIG. 13C, donor cell type as a percentage of total donor cells in spleen, FIG. 13D, donor cell type as a percentage of total donor cells in bone marrow; FIG. 13E, donor cell type as a percentage of total donor cells in thymus. Total donor chimerism.

FIG. 14A-D. Mice with long-term donor mixed chimerism generate in vitro response to third party, but not donor alloantigens. Spleens were harvested from long-term (>100 days) durable mixed chimeras and control mice that rejected donor BMCs shown in FIG. 12. Spleens were processed into single cell suspension, labeled with 2.5 μM CFSE, resuspended in DMEM, and 50×10⁶ CFSE labeled splenocytes were plated on a petri dish for 45 minutes at 37° C. to enrich lymphocytes. After 45 minutes non-adherent cells were collected, washed, and incubated (1×10⁵ cells) with irradiated (2000 cGy; 1×10⁵ cells) donor BALB/c or C3H splenocytes as stimulators in 96-well U-bottom titer plates in mixed lymphocyte reaction medium. Irradiated syngeneic C57BL/6 cells were used as controls. After 4 days, cells were stained with fluorochrome-labeled antibodies against rat CD4 and CD8, analyzed by flow cytometry gating on live cells, and the percentage of proliferating cells were graphed. FIG. 14A shows that CD4+ T cells from nonchimeric mice respond to irradiated splenocytes from both donor (BLAB/c) and third party (C3H) as stimulators. FIG. 14B shows that CD4+ T cells from long-term chimeric mice respond to irradiated splenocytes from third party (C3H), but not donor (BLAB/c) as stimulators. FIG. 14C shows that CD8+ T cells from nonchimeric mice respond to irradiated splenocytes from both donor (BLAB/c) and third party (C3H) as stimulators. FIG. 14D shows that CD8+ T cells from long-term chimeric mice respond to irradiated splenocytes from third party (C3H), but not donor (BLAB/c) as stimulators.

FIG. 15. FIG. 15 sets forth the human SA-FasL nucleotide construct and the linker sequence (SEQ ID NO: 1) between the streptavidin and FasL sequences.

FIG. 16A-B. FIGS. 16A and 16B set forth the human SA-FasL nucleotide sequence (SEQ ID NO:2) and protein sequence (SEQ ID NO: 3), respectively, of a fusion protein comprising streptavidin and FasL.

FIG. 17A-B. FIGS. 17A and 17B set forth the rat SA-FasL nucleotide sequence (SEQ ID NO:4) and protein sequence (SEQ ID NO: 5), respectively, of a fusion protein comprising streptavidin and FasL.

FIG. 18A-D. FIG. 15A sets forth the human SA-IL-2 Construct and linker sequences SEQ ID NO: 6 and SEQ ID NO: 10. FIGS. 18B and 18C set forth un-optimized DNA sequences SEQ ID NO: 7 and SEQ ID NO: 8, respectively. FIG. 18D sets forth human SA-IL-2 protein sequence (SEQ ID NO: 9).

### DETAILED DESCRIPTION

The present invention relates to the discovery that soluble SA-FasL, optionally together with IL-2, optionally together with FasL-decorated cells and/or IL-2-decorated cells, can be used to achieve long-term, specific immunosuppression.

CD8⁺ and CD4⁺ T effector cells, in particular CD4⁺ T cells, play a critical role in the initiation and perpetuation of various autoimmune diseases, including type I diabetes, rheumatoid arthritis, lupus, multiple sclerosis, and in foreign graft rejection, including rejection of allogeneic and xenogeneic grafts. T effector cells, therefore, represent an important target for immune modulation to prevent and treat these diseases. Under normal physiological conditions, T effector cells are kept in check by another class of T cells, designated as T regulatory cells. Mounting scientific evidence demonstrates that the disturbance of the physiological balance between T effector and T regulatory cells in favor of T effector cells is an underlying cause of many autoimmune diseases and foreign graft rejection. Approaches that target both T effector cells and T regulatory cells have significant therapeutic potential for reestablishing the physiological balance in autoimmunity, and for tilting the balance in favor of T regulatory cells in case of graft rejection.

Following antigen recognition and activation, T effector cells upregulate the Fas receptor on their surface and become sensitive to FasL-mediated apoptosis. Importantly FasL-mediated apoptosis is critical to the induction of self-tolerance and maintenance as deficiency in Fas or FasL is associated with massive autoimmunity both in humans and in rodents. This suggests that there are no compensatory mechanisms for this pathway, further emphasizing its importance as a target for immunomodulation.

Particular details of various embodiments of the invention are set forth below to illustrate certain aspects, but not to limit the scope of, the invention. It will be apparent to one of ordinary skill in the art that modifications and variations are possible without departing from the scope of the invention described herein. In the discussion that follows, specific embodiments of different aspects of the invention are described. It should be understood that any specific embodiment of one aspect may be used in conjunction with any specific embodiment of another aspect, even if every possible permutation and combination of specific embodiments is not expressly set forth.

### DEFINITIONS

Unless defined otherwise, all technical and scientific terms used herein have the same meaning as commonly understood by one of ordinary skill in the art to which the presently-disclosed subject matter belongs.

For the purposes of the present application, the following terms have these definitions:

As used herein “a” or “an” means one or more, unless specifically indicated to mean only one.

“Antigen” is used herein without limitation. Antigens include proteins, lipids, sugars, nucleic acids, chemical moieties, and other moieties that induce an immune response. Antigens include proteins, which may or may not be modified, such as by glycosylation or methylation, that are cyclized or bound to lipids, for example. In many embodiments, the antigens are present on the surface of cells, such as alloantigens. An antigen may also be a self antigen. Also suitable for use as an “antigen” in accordance with the present invention are peptides comprising antigenic portions of full-length proteins, such as peptides comprising a portion of a protein that induces an immune response, such as an immunogenic epitope.

“Immune cell” as used herein includes any cell that is involved in the generation, regulation or effect of the acquired or innate immune system. Immune cells include T cells such as CD4+ cells, CD8+ cells and various other T cell subets, B cells, natural killer cells, macrophages, monocytes and dendritic cells, and neutrophils.

“Autoantigen” means a self antigen, that despite being a normal tissue constituent, is the target of a humoral or cell-mediated immune response by the host, as in autoimmune disease.

“Surface” means a cell surface, the surface of a virus, the surface of a particle (e.g., the surface of a glass particle, the surface of a polysaccharide particle, the surface of a plastic particle), the surface of a phospholipid bilayer or the surface of a solid matrix, as well as the surface of hydrogels, scaffolds, beads, and the like.

“Cell surface” has its normal meaning in the art, comprising the phospholipid bilayer of a cell membrane and the molecules directly or indirectly associated with the bilayer.

As used herein, “decorated surface” means a surface to which is bound a chimeric protein, through interaction between the chimeric protein containing one member of a binding pair (e.g. SA) and the other member of the binding pair (e.g. biotin) that is bound to the surface (e.g. a cell membrane).

“Protein” means a protein or polypeptide that is native, non-native, synthetic or modified as by covalent binding.

Other terms are defined elsewhere herein. It is to be understood that each embodiment can be operated with every other embodiment.

### Proteins and Chimeric Proteins

Described herein are strategies for the induction of immune tolerance, some of which include the use of chimeric proteins which can be made by constructing chimeric cDNAs. In some embodiments, a chimeric protein comprises at least a functional portion of a member of a binding pair, such as streptavidin or avidin, operably linked to at least a functional portion of an apoptosis-inducing molecule, such as FasL, TNFa, TRAIL (Apo2 ligand), and TWEAK (Apo3 ligand) or the like. As used herein, the term “functional portion” of an apoptosis-inducing molecule means an apoptosis-inducing portion. As used herein, “FasL moiety” means at least the apoptosis-inducing moiety of FasL.

In particular embodiments, a chimeric protein comprises the extracellular domain of FasL as the apoptosis-inducing molecule, i.e. the FasL moiety. Independently, in particular embodiments, the chimeric protein comprises streptavidin (or core streptavidin) as the binding pair member. In specific embodiments, a chimeric protein comprises a streptavidin (or core streptavidin) moiety and a FasL moiety. In one embodiment, the chimeric FasL protein has the amino acid sequence of SEQ ID NO: 3.

The present invention uses IL-2. As used herein, “IL-2” includes chimeric IL-2, non chimeric IL-2 (such as may be obtained commercially) and proteins that contain the IL-2 moiety. “IL-2 moiety,” as used herein contains at least the part of IL-2 sufficient for binding to the IL-2 receptor and causing signaling. In some embodiments, a chimeric protein comprises at least a functional portion of a member of a binding pair, such as streptavidin or avidin, operably linked to at least a functional portion of IL-2, i.e. IL-2 moiety. In specific embodiments, a chimeric protein comprises a streptavidin (or core streptavidin) moiety and an IL-2 moiety. In one embodiment the chimeric IL-2 protein has the amino acid sequence of SEQ ID NO: 9. It is to be understood that these constructs are representative, and not limiting of chimeric proteins that can be used accordance with the present disclosure. As an example, a cell for the production of chimeric proteins as described herein may be the Drosophila system that is commercially available. Those skilled in the art of producing chimeric proteins will recognize that other expression systems and vectors are suitable for production of the chimeric proteins described herein, such as Escherichia coli, yeast and mammalian cell cultures.

“Binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-streptavidin, biotin-avidin, hormone-receptor, receptor-ligand, enzyme-substrate, lgG-protein A, antigen-antibody, and the like.

An exemplary binding pair is biotin and streptavidin (SA) or avidin. As used herein “biotin” includes biotin-containing moieties that are able to bind to surfaces, such as cell surfaces (including tumor cell surfaces), such as NHS-biotin and EZ-Link™ Sulfo-NHS-LC-Biotin (Pierce). Such protein reactive forms of biotin are available commercially.

The interaction between biotin and its binding partner, avidin or streptavidin, offers several advantages in the present context. For example, biotin has an extremely high affinity for both streptavidin (10¹³ M⁻¹) and avidin (10¹⁵ M⁻¹). This embodiment also is advantageous because conjugates comprising streptavidin or avidin can be further complexed with conjugates comprising biotin. Additionally, both streptavidin and avidin are tetrameric polypeptides that each bind four molecules of biotin. Conjugates comprising streptavidin or avidin therefore have a tendency to form tetramers and higher structures, and can form complexes with multiple biotin-containing moieties.

SA or avidin fragments which retain substantial binding activity for biotin, such as at least 50% or more of the binding affinity of native SA or avidin, respectively, also may be used. Such fragments include “core streptavidin” (“CSA”), a truncated version of the full-length streptavidin polypeptide which may include streptavidin residues 13-138, 14-138, 13-139 or 14-139. See, e.g., Pahler et al., 1987, J. Biol. Chem., 262: 13933-37. Other truncated forms of streptavidin and avidin that retain strong binding to biotin also may be used. See, e.g. Sano et al., 1995, J Biol Chem. 270(47): 28204-09 (describing core streptavidin variants 16-133 and 14-138) (U.S. Pat. No. 6,022,951). Mutants of streptavidin and core forms of strepavidin which retain substantial biotin binding activity or increased biotin binding activity also may be used. See, e.g., Chilcoti et al., 1995, Proc Natl Acad Sci USA. 92(5): 1754-58; Reznik et al., 1996, Nat Biotechnol. 14(8): 1007-11. For example, mutants with reduced immunogenicity, such as mutants mutated by site-directed mutagenesis to remove potential T cell epitopes or lymphocyte epitopes, can be used. See Meyer et al., 2001, Protein Sci. 10: 491-503. Likewise, mutants of avidin and core forms of avidin which retain substantial biotin binding activity or increased biotin binding activity also may be used. See Hiller et al., 1991, J. Biochem. 278: 573-85; Livnah et al., 1993, Proc Natl Acad Sci USA 90: 5076-80 (1993). For convenience, in the instant description, the terms “avidin” and “streptavidin” as used herein are intended to encompass biotin-binding fragments, mutants and core forms of these binding pair members. Avidin and streptavidin are available from commercial suppliers. Moreover, the nucleic acid sequences encoding streptavidin and avidin and the streptavidin and avidin amino acid sequences can be found, for example, in GenBank Accession Nos. X65082; X03591; NM_205320; X05343; Z21611; and Z21554.

Other mechanisms (e.g., other conjugation methods using, for example, other linking moieties or chemical or genetic cross-linking) can be used to provide linkages to bind a protein (e.g. FasL or IL-2) to a surface. Other mechanisms can be used to provide higher-order structures of immune co-stimulatory molecules, such as conjugates comprising dimers, trimers, tetramers and higher-order multimers of immune co-stimulatory molecules, which also will exhibit advantageous properties. Such conjugates are included within the scope of this invention.

As used herein, the existing of a binding moiety permits the chimeric protein to bind to e.g. a cell surface. As used herein, a cell to which is bound FasL, IL-2 and the like may be said to be “decorated” with FasL, IL-2, etc. Likewise, the FasL on a cell would be a “membrane bound FasL,” “bound FasL”, or “mFasL”; and the IL-2 on a cell would be “membrane bound IL-2”, “bound IL-2” or “mIL-2”

A chimeric protein may also be unbound to a surface. Such a chimeric protein may be said to be “soluble.” As used herein “soluble FasL” or “sFasL” means a molecule containing the FasL moiety that is not bound to a surface (such as a cell). The FasL may be a chimeric FasL. It should be noted that “soluble FasL” as used herein contains the apoptotic extracellular moiety of FasL, and is distinct from an unbound molecule derived from FasL that is unable to induce apoptosis.

A “soluble IL-2” or “sIL-2” as used herein means that the IL-2 is not bound not bound to a surface (such as a cell). The soluble IL-2 may be a chimeric IL-2, or may be non-chimeric IL-2.

### Cells

Certain embodiments described herein use or comprise a cell, tissue or organ. In particular embodiments, the cell is a mammalian cell. In embodiments related to graft rejection, the cell may be of the same type as the cell of the graft to be transplanted. Additionally or alternatively, the cell may be of a different type from the graft but from the same donor, to induce tolerance to the donor graft. For example, FasL-decorated donor cells can be used to induce immune tolerance in the subject who has received, is receiving, or will receive a graft from the same donor. The administration of donor tissue, organs or cells is then more likely to be tolerated by the subject.

To illustrate, a subject may be administered pancreatic islet cells to treat diabetes. To prevent rejection, the subject may be administered pancreatic islet cells that are decorated with FasL. Additionally or alternatively, the subject may be administered a different type of FasL-decorated donor cells, such as splenocytes, PBMCs, bone marrow cells, mesenchymal stem cells, hematopoietic stem cells, stem cells, induced pluripotent stem cells, dendritic cells, cardiac myocytes, and vascular cells, etc., to induce immune tolerance to the donor's cells. Depending on the condition being treated, the subject may be administered FasL-decorated cells, such as splenocytes, PBMCs, bone marrow cells, mesenchymal stem cells, hematopoietic stem cells, stem cells, induced pluripotent stem cells, dendritic cells, cardiac myocytes, and vascular cells, etc., to treat the condition, and/or may be administered decorated or undecorated cells to treat the condition and one or more different types of FasL-decorated cells to induce immune tolerance to the donor's cells.

In some embodiments, the alloantigen is a pancreatic islet cell, a tissue, or an organ. In some embodiments, a cell used to treat the subject (or provided in the medicament) is selected from the group consisting of islet cells, bone marrow cells, hematopoietic stem cells, stem cell, induced pluripotent stem cell, human beta cell products, hepatocytes, dendritic cells, macrophages, endothelial cells, mesenchymal stem cell, and immune cells, including T cells. In further specific embodiments, the cell is part of a tissue or organ.

In some embodiments, the antigen is a self antigen. In some embodiments, a cell used to treat the subject (or provided in the medicament) is engineered to express an autoantigen, or is decorated with an autoantigen. For example, dendritic cells can be pulsed with autoantigen and thereby display the antigen on the surface.

In some embodiments, cells from the subject are used to induce immune tolerance to self that has been interrupted in autoimmune disease. That is, the “donor” and “recipient” may be the same subject. Exemplary cells suitable for use in these embodiments include mobilized hematopoietic stem cells, PBMCs, dendritic cells, and the like. In some embodiments, the cells are chosen from those that naturally express self antigens that are targeted in the autoimmune disease. For example, type I diabetes is an autoimmune disease wherein the body reacts and rejects pancreatic islet (β) cells. In early stages of diabetes, before all islet cells are rejected, it can be possible to induce tolerance to islet cells and thereby prevent the progression of diabetes.

In specific embodiments, host-derived cells are decorated with SA-FasL and/or SA-IL-2 and used either alone or in combination with soluble SA-FasL and/or IL-2. For example, host-derived cells can be decorated with SA-FasL and used with soluble SA-FasL, and optionally further with IL-2. In other embodiments, a dendritic cell is pulsed with autoantigen and decorated with SA-FasL and/or SA-IL-2 and used with soluble SA-FasL and/or soluble IL-2, to induce tolerance to the autoantigen. For example, dendritic cells can be pulsed with autoantigen and decorated with SA-FasL and used with soluble SA-FasL, and optionally further with IL-2.

In other embodiments cells, such as bone marrow, PBMCs, DCs, etc., are decorated with autoantigen and also decorated with SA-FasL, and optionally further with SA-IL2, and administered with soluble SA-FasL, and optionally further with IL-2 to treat autoimmunity.

In specific embodiments, the autoantigen is associated with diabetes, rheumatic fever, multiple sclerosis or lupus.

### Medicaments and Compositions

Some aspects of the invention relates to the surprising discovery that soluble SA-FasL, optionally further with IL-2, can be used to achieve long-term, specific immunosuppression. Further particular aspects of the invention relates to the surprising discovery that soluble SA-FasL, optionally further with IL-2, can be used with FasL-decorated cells to achieve long term and specific immunosuppression.

In some embodiments, there are provided medicaments for use in inducing immune tolerance in a subject in need thereof, comprising (i) a chimeric FasL protein comprising a FasL moiety and a streptavidin or avidin moiety and (ii) an IL-2 protein. In any embodiments, the chimeric FasL protein and IL-2 protein may be provided in separate compositions or in the same composition.

Unless otherwise specified, in any embodiments, the chimeric FasL protein may be selected from soluble chimeric FasL proteins comprising a FasL moiety and a streptavidin or avidin moiety and chimeric FasL-decorated cells comprising a chimeric FasL protein comprising a FasL moiety and a streptavidin or avidin moiety bound to a cell surface via a biotin moiety on the cell surface. One embodiment of chimeric FasL protein has the amino acid sequence of SEQ ID NO: 3.

Unless otherwise specified, in any embodiments, the IL-2 protein may be selected from soluble IL-2 proteins, soluble chimeric IL-2 proteins comprising an IL-2 moiety and a streptavidin or avidin moiety; and chimeric IL-2-decorated cells comprising a chimeric IL-2 protein comprising an IL-2 moiety and a streptavidin or avidin moiety bound to a cell surface via a biotin moiety on the cell surface. One embodiment of chimeric IL-2 protein has the amino acid sequence of SEQ ID NO: 9.

In some embodiments, the medicament comprises soluble chimeric FasL protein and soluble IL-2 protein or soluble chimeric IL-2 protein. In other embodiments, the medicament comprises chimeric FasL-decorated cells and soluble IL-2 protein or soluble chimeric IL-2 protein. In another, the medicament comprises soluble chimeric FasL protein and chimeric IL-2-decorated cells. In another, the medicament comprises chimeric FasL-decorated cells and chimeric IL-2-decorated cells. In specific embodiments, a medicament for use in inducing immune tolerance may comprise soluble chimeric FasL protein comprising a FasL moiety and a streptavidin or avidin moiety.

The medicaments are effective for inducing immunomodulation in subjects in need of such immunomodulation, and can be used in such methods. For example, when the subject is in need of treatment for type 1 diabetes, the decorated cells, if present, may be splenocytes, PBMC, bone marrow cells, mesenchymal stem cells, hematopoietic stem cells, stem cells, induced pluripotent stem cells, dendritic cells, and dendritic cells pulsed with autoantigens, and others as discussed above. When the subject is in need of the treatment or prevention of allograft rejection, the decorated cells, if present, may be cells from the allograft donor, such as cells selected from the group consisting of allograft bone marrow cells, allograft cardiac myocytes and allograft vascular cells, or other cells from the allograft donor as discussed above.

One embodiment of the presently-disclosed subject matter is directed to a pharmaceutical composition or medicament comprising (a) cells comprising biotin on their surface, (b) a chimeric protein comprising (i) an apoptosis-inducing FasL moiety and (ii) a member of a binding pair selected from the group consisting of avidin and streptavidin moieties; wherein the chimeric protein of (b) is bound through the avidin or streptavidin moiety to biotin on the surface of said cells of (a), and (c) a chimeric protein comprising (i) an apoptosis-inducing FasL moiety and (ii) a member of a binding pair selected from the group consisting of avidin and streptavidin moieties, wherein the chimeric protein of (c) is in soluble form. In some embodiments, the chimeric proteins of (b) and (c) form tetramers and/or oligomers. In some embodiments, the composition comprises an amount of these components effective to induce immune tolerance when administered to a subject in need thereof.

Another embodiment of the presently-disclosed subject matter is directed to a pharmaceutical composition or medicament comprising (a) cells comprising biotin on their surface, (b) a chimeric protein comprising (i) an apoptosis-inducing FasL moiety and (ii) a member of a binding pair selected from the group consisting of avidin and streptavidin moieties; wherein the chimeric protein of (b) is bound through the avidin or streptavidin moiety to biotin on the surface of said cells of (a), and (c) soluble IL-2. In some embodiments, the composition comprises an amount of these components effective to induce immune tolerance when administered to a subject in need thereof.

An additional embodiment is directed to a pharmaceutical composition or medicament comprising (a) cells comprising biotin on its surface, (b) a chimeric protein comprising (i) an apoptosis-inducing FasL moiety and (ii) a member of a binding pair selected from the group consisting of avidin and streptavidin moieties; (c) a chimeric protein comprising (i) an IL-2 moiety and (ii) a member of a binding pair selected from the group consisting of avidin and streptavidin moieties wherein the chimeric proteins of (b) and (c) are bound through the avidin or streptavidin moiety to biotin on the surface of said cells of (a). In some embodiments, the composition comprises an amount of these components effective to induce immune tolerance when administered to a subject in need thereof.

In some embodiments, the FasL moiety is positioned C-terminal to the binding pair member moiety. In other embodiments, the FasL moiety is positioned N-terminal to the binding pair member moiety. In any embodiments, the member of a binding pair may be streptavidin or core streptavidin. In some embodiments, the chimeric FasL protein forms tetramers and/or oligomers.

The FasL moiety may be wild type FasL (wtFasL includes any mammalian FasL, including rat, mouse, or human wild type FasL), modified FasL (mFasL includes any mammalian FasL modified to be stably expressed on a cell surface), or soluble (sFasL soluble includes the extracellular portion of any mammalian FasL). In any embodiments, a construct comprising a FasL moiety can further include one or more linkers, zinc zippers, can be pegylated, etc.

In some embodiments, a construct comprising a FasL moiety is capable of binding through the FasL moiety to a cell expressing a death receptor. If the construct is bound via a member of a binding pair to a cell, the construct may be capable of binding to a further cell expressing a death receptor through the FasL moiety.

In any embodiments of a construct comprising IL-2, the construct may be a soluble IL-2 construct, such as a chimeric protein comprising (i) an IL-2 moiety and (ii) a member of a binding pair selected from the group consisting of avidin and streptavidin moieties. In any embodiments, the member of a binding pair may be streptavidin or core streptavidin. The IL-2 moiety may be positioned C-terminal or N-terminal to the binding pair member moiety. In some embodiments, the chimeric IL-2 protein forms tetramers and/or oligomers.

The IL-2 moiety may be wtIL-2 (including any mammalian IL-2, including rat, mouse, or human wild type IL-2 molecule), mIL-2 (including any mammalian IL-2 modified to be stably expressed on the cell surface), or sIL-2 (including the soluble extracellular portion of any mammalian IL-2). In any embodiments, the construct comprising an IL-2 moiety can further include one or more linkers, zinc zippers, can be pegylated, etc.

In some embodiments, a construct comprising an IL-2 moiety is capable of binding through the IL-2 moiety to a cell expressing an IL-2 receptor. If the construct is bound via a member of a binding pair to a cell, the construct may be capable of binding to a further cell expressing an IL-2 receptor through the FasL moiety.

In some embodiments, any pharmaceutical composition or medicament described herein may further comprise an alloantigen and/or an autoantigen. In some embodiments, the alloantigen is a cell. In particular embodiments, the cell is a mammalian cell. In certain embodiments, the alloantigen is a pancreatic islet cell, a tissue, or an organ. In other embodiments, the cell of the pharmaceutical composition is selected from the group consisting of islet cells, bone marrow cells, hematopoietic stem cells, stem cell, induced pluripotent stem cell, human beta cell products, hepatocytes, dendritic cells, PBMC, macrophages, endothelial cells, mesenchymal stem cell, and immune cells, including T cells. In even further embodiments, the cell is part of a tissue or organ. Other embodiments of cells are discussed above.

A further embodiment of the presently-disclosed subject matter is directed to medicaments comprising (a) a chimeric protein comprising (i) an apoptosis-inducing FasL moiety and (ii) a member of a binding pair selected from the group consisting of avidin and streptavidin moieties, wherein the chimeric protein of (a) is in soluble form, and (b) soluble IL-2. In some embodiments, the composition comprises an amount of these components effective to induce immune tolerance when administered to a subject in need thereof.

The chimeric proteins may be provided in separate compositions, or in a single composition. Each composition may further comprise a pharmaceutically acceptable carrier, excipient or diluent, as known in the art. Consistent with the conventions in the art, a pharmaceutically acceptable carrier may be a material that can be used as a vehicle for the active agents in the composition because the carrier material is inert or otherwise medically acceptable, as well as compatible with the active agent(s), in the context of formulation, storage, and administration. A pharmaceutically acceptable carrier can contain conventional pharmaceutical additives like diluents and preservatives.

### Methods

The invention also includes methods for inducing immune tolerance. In some embodiments, the method of inducing immune tolerance in a subject in need thereof comprises administering to the individual (i) a chimeric FasL protein comprising a FasL moiety and a streptavidin or avidin moiety and (ii) an IL-2 protein. The FasL protein and the IL-2 protein may be administered together or separately, in any order. In any such embodiments, the FasL may be selected from soluble chimeric FasL proteins comprising a FasL moiety and a streptavidin or avidin moiety and chimeric FasL-decorated cells comprising a chimeric FasL protein comprising a FasL moiety and a streptavidin or avidin moiety bound to a cell surface via a biotin moiety on the cell surface, where the FasL moiety may be any FasL moiety discussed above.

The IL-2 protein may be selected from the group consisting of soluble IL-2 proteins, soluble chimeric IL-2 proteins comprising an IL-2 moiety and a streptavidin or avidin moiety; and chimeric IL-2-decorated cells comprising a chimeric IL-2 protein comprising an IL-2 moiety and a streptavidin or avidin moiety bound to a cell surface via a biotin moiety on the cell surface, where the IL-2 moiety may be any IL-2 moiety discussed above.

Such methods may be practiced on a subject in need of treatment for type 1 diabetes, wherein the decorated cells, if present, are PBMCs, splenocytes, bone marrow cells, mesenchymal stem cells, hematopoietic stem cells, stem cells, dendritic cells, or induced pluripotent stem cells, or other suitable cells such as discussed above. For example, such a method may comprise administering to the subject in need thereof:
(a) chimeric FasL-decorated pancreatic islet cells;
(b)(i) soluble chimeric FasL protein and (ii) soluble IL-2 protein or soluble chimeric IL-2 protein, in amounts effective to induce immune tolerance to pancreatic islet cells; and
(c) optionally, administering an immunosuppressant, such as rapamycin.

In further embodiments, the subject is in need of the treatment or prevention of allograft rejection and the decorated cells, if present, are cells from the allograft donor, such as cells selected from the group consisting of allograft bone marrow cells, allograft cardiac myocytes and allograft vascular cells, or other cells from the allograft donor, as discussed above. For example, such a method of treating or preventing allograft rejection in a subject in need thereof, may comprise:
(a) administering chimeric FasL-decorated cells from the allograft donor, such as cells selected from the group consisting of allograft bone marrow cells, allograft cardiac myocytes and allograft vascular cells, or other cells from the allograft donor;
(b) administering (i) soluble chimeric FasL protein and (ii) soluble IL-2 protein or soluble chimeric IL-2 protein, in amounts effective to induce immune tolerance to the allograft cells; and
optionally, administering an immunosuppressant, such as rapamycin.

In specific embodiments of any of these methods, the allograft may be decorated with one or both FasL and IL-2.

In specific embodiments of any of these methods, the chimeric FasL protein may have the amino acid sequence of SEQ ID NO: 3. Independently, in specific embodiments of any of these methods, the chimeric IL-2 may have the amino acid sequence of SEQ ID NO: 9.

Further embodiments are directed to methods of inducing immune modulation in a mammal in need thereof comprising administering an effective amount of any pharmaceutical composition or medicament as described herein to a mammal having a condition which is alleviated by the apoptosis of activated pathogenic lymphocytes. In certain embodiments of a method of inducing immune modulation in a mammal in need thereof, the mammal has a condition which is alleviated by the apoptosis of activated pathogenic lymphocytes and the induction/expansion of protective lymphocytes, such as T regulatory cells. In other embodiments, the mammal can have a genetically inherited hematopoietic metabolic disorder or cancer. In certain embodiments of a method of inducing immune modulation in a mammal in need thereof, the mammal has a condition selected from the group consisting of asthma, allergy, food poisoning, autoimmunity, and transplantation of allogencic or xenogeneic cells, tissues, and organs. For example, bone marrow transplantation is a critical tool in the treatment of leukemia and other cancers. Stem cell transplants show promise to repair damaged or degenerated tissue, and therefore tolerance to donor stem cells is advantageous, Other situations where tolerance to donor tissues, organs or cells is advantageous include heart transplants, kidney transplants, liver transplants, vascular transplants, skin transplants, and blood transfusions. The methods described herein can be used in such contexts.

In more specific embodiments, the autoimmune condition is selected from the group consisting of diabetes, multiple sclerosis, lupus erythematosis, sarcoidsis, Sjögren's syndrome, polymyalgia rheumatica, ankylosing spondylitis, alopecia areata, and rheumatoid arthritis. autoimmune hematological disorders (including e.g. hemolytic anaemia, aplastic anaemia, pure red cell anaemia and idiopathic thrombocytopenia), systemic lupus erythematosus, polychondritis, sclerodoma, Wegener granulomatosis, dermatomyositis, chronic active hepatitis, myasthenia gravis, psoriasis, Steven-Johnson syndrome, idiopathic sprue, (autoimmune) inflammatory bowel disease (including e.g. ulcerative colitis and Crohn's disease), endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis, glomerulonephritis (with and without nephrotic syndrome, e.g. including idiopathic nephrotic syndrome or minimal change nephropathy) and juvenile dermatomyositis. Accumulating data indicate that many chronic diseases, such as Type 2 diabetes atherosclerosis, may be caused by immune dysregulation, i.e. pathogenic/regulatory cell imbalance. Autoimmune and inflammatory conditions of the skin are also considered to be amenable to treatment and prevention using the synergistic combination of the invention, e.g., psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphigus, epidermolysis bullosa acquisita, and other inflammatory or allergic conditions of the skin, as are inflammatory conditions of the lungs and airways including asthma, allergies, and pneumoconiosis.

When preventing or treating autoimmunity in a subject in need thereof, the method may comprise (a) administering to the subject cells decorated with chimeric FasL and optionally decorated with chimeric IL-2, and (b) administering (i) soluble chimeric FasL protein and (ii) soluble IL-2 protein or soluble chimeric IL-2 protein, in amounts effective to induce immune tolerance to the autoantigen; wherein the cells are selected from (i) a cell expressing an autoantigen (ii) a cell decorated with an autoantigen and (ii) a dendritic cell pulsed with the autoantigen. In particular embodiments, the cell expressing an autoantigen or the cell decorated with the autoantigen is obtained from the patient, and is selected from bone marrow cells, dendritic cells, PBMC, hematopoietic stem cells, or other cells as discussed above.

In accordance with further embodiments, there are provided methods of treating or preventing autoimmunity in a subject in need thereof, comprising (a) administering an autoantigen presented on a cell selected from the group consisting of (i) a cell expressing the autoantigen (ii) a cell decorated with the autoantigen and (iii) a dendritic cell pulsed with the autoantigen, wherein the cell optionally is decorated with chimeric FasL and/or chimeric IL-2, and (b) administering (i) soluble chimeric FasL protein and (ii) soluble IL-2 protein or soluble chimeric IL-2 protein, in amounts effective to induce immune tolerance to the autoantigen. In particular embodiments, the cell is obtained from the subject. In specific embodiments, the cell is selected from bone marrow cells, dendritic cells, PBMC, hematopoietic stem cells, and mesenchymal stem cells, including any of such cells obtained from the subject.

In specific embodiments of any of these methods, the chimeric FasL protein may have the amino acid sequence of SEQ ID NO: 3. Independently, in specific embodiments of any of these methods, the chimeric IL-2 may have the amino acid sequence of SEQ ID NO: 9.

In any embodiments, the method may further comprise administering an effective amount of an immunomodulatory drug. Exemplary immunomodulatory drugs include rapamycin and cyclophosamide. Other non-limiting examples include busulfan, fludarabine, methotrexate, sulfasalazine, hydroxychloroquine, azathioprine, tocilizumab, etanercept, adalimumab, anakinra, abatacept, rituximab, certolizumab, golimumab, cyclosporine, dexamethasone, methylprednisolone, predinisone, and triamcinolone.

Absolute dosages of the compounds will vary depending on the individual, the route of administration and the nature and severity of the condition to be treated. Generally speaking, the dosages of FasL and IL-2 reported in the examples, as used in mice, can be converted to human dosages according to the following table:

| | | TO | | | | |
|---|---|---|---|---|---|---|
| | | Mouse | Rat | Monkey | Dog | Man |
| | | 20 g | 150 g | 3 kg | 8 kg | 60 kg |
| FROM | Mouse | 1 | ½ | ¼ | ⅙ | 1/12 |
| | Rat | 2 | 1 | ½ | ¼ | 1/7 |
| | Monkey | 4 | 2 | 1 | ⅗ | ⅓ |
| | Dog | 6 | 4 | 1⅔ | 1 | ½ |
| | Man | 12 | 7 | 3 | 2 | 1 |

Furthermore, the dosage necessary to induce apoptosis may depend on the affinity and specificity of the apoptotic agent. The amount of apoptotic agent administered also may vary with the number of cells being treated.

Generally speaking, the methods described herein using FasL may include administering FasL doses of from less than about 0.2 μg/day/patient to at least about 10 μg/day/patient, or more, based on the FasL moiety. For example, methods described herein may be carried out using daily doses of FasL at amounts of less than about 0.2 μg/day/patient, about 0.2 μg/day/patient, about 0.5 μg/day/patient, about 1 μg/day/patient, about 1.5 μg/day/patient, about 2 μg/day/patient, about 2.5 μg/day/patient, about 3 μg/day/patient, about 3.5 μg/day/patient, about 4 μg/day/patient, about 4.5 μg/day/patient, about 5 μg/day/patient, or more.

Generally speaking, the methods described herein using IL-2 may include administering IL-2 at daily doses ranging from less than about 5000 IU/day/patient to at least about 30000000 IU/day/patient, or more, based on the IL-2 moiety. For example, methods described herein may be carried out using daily IL-2 doses of less than about 5000 IU/day/patient, about 5000 IU/day/patient, about 10000 IU/day/patient, about 25000 IU/day/patient, about 50000 IU/day/patient, about 100000 IU/day/patient, 200000 IU/day/patient, 500000 IU/day/patient, 1000000 IU/day/patient, 2000000 IU/day/patient, 30000000 IU/day/patient, or more.

As noted above, these dosages are illustrative only. The dosage and schedule of administration may vary within and even from these ranges depending on the aim of the treatment, the disease to be treated, the specific subject, etc.

In some embodiments, the treatment is administered over a course of several days, for example over 1-30 days, including from about 7 to about 15 days, for about 1 week, or for about 2 weeks. As with dosage, the duration of treatment may vary within and even from these ranges depending on the aim of the treatment, the disease to be treated, the specific subject, etc.

### Additional Specific Embodiments

Specific embodiments of the compositions and methods described herein include:
E1. A pharmaceutical composition comprising an effective amount of: (a) a cell comprising biotin on its surface, (b) a chimeric protein comprising (i) an apoptosis-inducing FasL moiety and (ii) a member of a binding pair selected from the group consisting of avidin and streptavidin moieties; wherein the chimeric protein of (b) is bound through the avidin or streptavidin moiety to biotin on the surface of said cells of (a), and (c) a chimeric protein comprising (i) an apoptosis-inducing FasL moiety and (ii) a member of a binding pair selected from the group consisting of avidin and streptavidin moieties, wherein the chimeric protein of (c) is in soluble form.
E2. The pharmaceutical composition of embodiment 1, wherein the apoptosis-inducing FasL moiety of (b) and (c) are wtFasL, mFasL, or sFasL.
E3. The pharmaceutical composition of embodiment 1, wherein the chimeric proteins of (b) and (c) bind through the FasL moiety to a further cell expressing a death receptor.
E4. The pharmaceutical composition of embodiment 1, wherein the chimeric proteins of (b) and (c) form tetramers and/or oligomers.
E5. The pharmaceutical composition of embodiment 1, wherein the member of a binding pair of (b) and (c) is streptavidin.
E6. The pharmaceutical composition of embodiment 5, wherein the streptavidin is core streptavidin.
E7. The pharmaceutical composition of embodiment 2, wherein the FasL of (b) and (c), is wtFasL, mFasL, or sFasL.
E8. The pharmaceutical composition of embodiment 1, wherein the apoptosis-inducing FasL moiety of (b) and (c) is positioned C-terminal or N-terminal to the binding pair member moiety.
E9. The pharmaceutical composition of embodiment 1, wherein said chimeric proteins of (b) and (c) consist essentially of (i) an apoptosis-inducing FasL moiety and (ii) a member of a binding pair selected from the group consisting of avidin and streptavidin moieties.
E10. The pharmaceutical composition of embodiment 1, wherein the cell of (a) is selected from a splenocyte, a PBMC, and a bone marrow cell.
E11. The pharmaceutical composition of embodiment 1, further comprising an alloantigen or autoantigen.
E12. The pharmaceutical composition of embodiment 11, wherein the alloantigen is a cell.
E13. The pharmaceutical composition of embodiment 11, wherein the alloantigen is a pancreatic islet cell, a tissue, or an organ.
E14. The pharmaceutical composition of embodiment 1, wherein the cell of (a) is selected from the group consisting of islet cells, bone marrow cells, hematopoietic stem cells, stem cell, induced pluripotent stem cell, human beta cell products, hepatocytes, dendritic cells, mesenchymal cells, macrophages, endothelial cells, and T cells.
E15. The pharmaceutical composition of embodiment 1, wherein the cell of (a) is part of a tissue or organ.
E16. A method of inducing immune modulation in a mammal in need thereof comprising administering an effective amount of the pharmaceutical composition of embodiment 1 to a mammal having a condition which is alleviated by the apoptosis of activated pathogenic lymphocytes.
E17. The method of embodiment 16, wherein the mammal has a condition selected from the group consisting of asthma, allergy, food poisoning, autoimmunity, and transplantation of allogeneic or xenogeneic tissue.
E18. The method of embodiment 17, wherein the autoimmune condition is selected from the group consisting of diabetes, multiple sclerosis, lupus erythematosis, sarcoidsis, and rheumatoid arthritis.
E19. The method of embodiment 16, further comprising administering an effective amount of rapamycin.
E20. The method of embodiment 19, further comprising administering an effective amount of cyclophosamide.
E21. A pharmaceutical composition comprising an effective amount of: (a) a cell comprising biotin on its surface, (b) a chimeric protein comprising (i) an apoptosis-inducing FasL moiety and (ii) a member of a binding pair selected from the group consisting of avidin and streptavidin moieties; wherein the chimeric protein of (b) is bound through the avidin or streptavidin moiety to biotin on the surface of said cells of (a), and (c) soluble IL-2.
E22. The pharmaceutical composition of embodiment 21, wherein the apoptosis-inducing FasL moiety of (b) is wtFasL, mFasL, or rsFasL.
E23. The pharmaceutical composition of embodiment 21, wherein the soluble IL-2 is a chimeric protein comprising (i) an IL-2 moiety and (ii) a member of a binding pair selected from the group consisting of avidin and streptavidin moieties.
E24. The pharmaceutical composition of embodiment 23, wherein the IL-2 moiety of (c) is wtIL-2 or sIL-2.
E25. The pharmaceutical composition of embodiment 21, wherein the chimeric protein of (b) binds through the FasL moiety to a further cell expressing a death receptor.
E26. The pharmaceutical composition of embodiment 23, wherein the chimeric IL-2 protein of (c) binds through the IL-2 moiety to a further cell expressing an IL-2 receptor.
E27. The pharmaceutical composition of embodiment 21, wherein the chimeric protein of (b) forms tetramers and/or oligomers.
E28. The pharmaceutical composition of embodiment 23, wherein the chimeric IL-2 protein forms tetramers and/or oligomers.
E29. The pharmaceutical composition of embodiment 21, wherein the member of a binding pair of (b) and is streptavidin.
E30. The pharmaceutical composition of embodiment 23, wherein the member of a binding pair of the chimeric IL-2 protein is streptavidin.
E31. The pharmaceutical composition of embodiment 29, wherein the streptavidin is core streptavidin.
E32. The pharmaceutical composition of embodiment 30, wherein the streptavidin is core streptavidin.
E33. The pharmaceutical composition of embodiment 22 wherein the FasL of (b), is mFasL.
E34. The pharmaceutical composition of embodiment 21, wherein the apoptosis-inducing FasL moiety of (b) is positioned C-terminal to the binding pair member moiety.
E35. The pharmaceutical composition of embodiment 23, wherein the IL-2 moiety is positioned N-terminal to the binding pair member moiety.
E36. The pharmaceutical composition of embodiment 21, wherein said chimeric protein of (b) consists essentially of (i) an apoptosis-inducing FasL moiety and (ii) a member of a binding pair selected from the group consisting of avidin and streptavidin moieties.
E37. The pharmaceutical composition of embodiment 23, wherein said chimeric IL-2 protein consists essentially of (i) an IL-2 moiety and (ii) a member of a binding pair selected from the group consisting of avidin and streptavidin moieties.
E38. The pharmaceutical composition of embodiment 21, wherein the cell of (a) is a splenocyte or peripheral blood mononuclear cell (PBMC).
E39. The pharmaceutical composition of embodiment 21, further comprising an alloantigen or autoantigen.
E40. The pharmaceutical composition of embodiment 39, wherein the alloantigen is a cell.
E41. The pharmaceutical composition of embodiment 39, wherein the alloantigen is a pancreatic islet cell, a tissue, or an organ.
E42. The pharmaceutical composition of embodiment 21, wherein the cell of (a) is selected from the group consisting of islet cells, bone marrow cells, hematopoietic stem cells, human beta cell products, hepatocytes, dendritic cells, mesenchymal cells, macrophages, endothelial cells, and T-cells.
E43. The pharmaceutical composition of embodiment 21, wherein the cell of (a) is part of a tissue or organ.
E44. A method of inducing immune modulation in a mammal in need thereof comprising administering an effective amount of the pharmaceutical composition of embodiment 21 to a mammal having a condition which is alleviated by the apoptosis of activated pathogenic lymphocytes.
E45. The method of embodiment 44, wherein the mammal has a condition selected from the group consisting of asthma, allergy, food poisoning, autoimmunity, and transplantation of allogeneic or xenogeneic tissue.
E46. The method of embodiment 45, wherein the autoimmune condition is selected from the group consisting of diabetes, multiple sclerosis, lupus erythematosis, sarcoidsis, and rheumatoid arthritis.
E47. The method of embodiment 44, further comprising administering an effective amount of a chimeric protein comprising (i) an apoptosis-inducing FasL moiety and (ii) a member of a binding pair selected from the group consisting of avidin and streptavidin moieties, wherein the chimeric protein is in soluble form.
E48. The method of embodiment 47, further comprising administering an effective amount of rapamycin.
E49. The method of embodiment 48, further comprising administering an effective amount of cyclophosphamide.
E50. A pharmaceutical composition comprising an effective amount of: (a) a chimeric protein comprising (i) an apoptosis-inducing FasL moiety and (ii) a member of a binding pair selected from the group consisting of avidin and streptavidin moieties, wherein the chimeric protein of (a) is in soluble form, and (b) soluble IL-2.
E51. The pharmaceutical composition of embodiment 50, wherein the apoptosis-inducing FasL moiety of (a) is wtFasL, mFasL, or sFasL.
E52. The pharmaceutical composition of embodiment 50, wherein the soluble IL-2 is a chimeric protein comprising (i) an IL-2 moiety and (ii) a member of a binding pair selected from the group consisting of avidin and streptavidin moieties.
E53. The pharmaceutical composition of embodiment 52, wherein the IL-2 moiety of (b) is wtIL-2, m-IL-2, or sIL-2.
E54. The pharmaceutical composition of embodiment 50 wherein the chimeric protein of (a) binds through the FasL moiety to a further cell expressing a death receptor.
E55. The pharmaceutical composition of embodiment 52, wherein the chimeric IL-2 protein of binds through the IL-2 moiety to a further cell expressing an IL-2 receptor.
E56. The pharmaceutical composition of embodiment 50, wherein the chimeric protein of (a) forms tetramers and/or oligomers.
E57. The pharmaceutical composition of embodiment 52, wherein the chimeric IL-2 protein forms tetramers and/or oligomers.
E58. The pharmaceutical composition of embodiment 50, wherein the member of a binding pair of (a) is streptavidin.
E59. The pharmaceutical composition of embodiment 52, wherein the member of a binding pair of the chimeric IL-2 protein is streptavidin.
E60. The pharmaceutical composition of embodiment 58, wherein the streptavidin is core streptavidin.
E61. The pharmaceutical composition of embodiment 59, wherein the streptavidin is core streptavidin.
E62. The pharmaceutical composition of embodiment 51 wherein the FasL of (a) is sFasL.
E63. The pharmaceutical composition of embodiment 50, wherein the apoptosis-inducing FasL moiety of (a) is positioned C-terminal to the binding pair member moiety.
E64. The pharmaceutical composition of embodiment 52, wherein the IL-2 moiety is positioned N-terminal to the binding pair member moiety.
E65. The pharmaceutical composition of embodiment 50, wherein said chimeric protein of (a) consists essentially of (i) an apoptosis-inducing FasL moiety and (ii) a member of a binding pair selected from the group consisting of avidin and streptavidin moieties.
E66. The pharmaceutical composition of embodiment 52, wherein said chimeric IL-2 protein consists essentially of (i) an IL-2 moiety and (ii) a member of a binding pair selected from the group consisting of avidin and streptavidin moieties.
E67. The pharmaceutical composition of embodiment 50, further comprising an alloantigen or autoantigen.
E68. The pharmaceutical composition of embodiment 67, wherein the alloantigen is a cell.
E69. The pharmaceutical composition of embodiment 67, wherein the alloantigen is a pancreatic islet cell, a tissue, or an organ.
E70. A method of inducing immune modulation in a mammal in need thereof comprising administering an effective amount of the pharmaceutical composition of embodiment 50 to a mammal having a condition which is alleviated by the apoptosis of activated pathogenic lymphocytes.
E71. The method of embodiment 70, wherein the mammal has a condition selected from the group consisting of asthma, allergy, food poisoning, autoimmunity, and transplantation of allogeneic or xenogeneic tissue.
E72. The method of embodiment 71, wherein the autoimmune condition is selected from the group consisting of diabetes, multiple sclerosis, lupus erythematosis, sarcoidsis, and rheumatoid arthritis.
E73. The method of embodiment 70, further comprising administering an effective amount (a) a cell comprising biotin on its surface, (b) a chimeric protein comprising (i) a IL-2 moiety and (ii) a member of a binding pair selected from the group consisting of avidin and streptavidin moieties; wherein the chimeric protein of (b) is bound through the avidin or streptavidin moiety to biotin on the surface of said cells of (a).
E74. The method of embodiment 73, further comprising administering an effective amount of rapamycin.
E75. The method of embodiment 74, further comprising administering an effective amount of cyclophosphamide.
E76. A pharmaceutical composition comprising an effective amount of: (a) a cell comprising biotin on its surface, (b) a chimeric protein comprising (i) an apoptosis-inducing FasL moiety and (ii) a member of a binding pair selected from the group consisting of avidin and streptavidin moieties; (c) a chimeric protein comprising (i) an IL-2 moiety and (ii) a member of a binding pair selected from the group consisting of avidin and streptavidin moieties wherein the chimeric proteins of (b) and (c) are bound through the avidin or streptavidin moiety to biotin on the surface of said cells of (a).
E77. The pharmaceutical composition of embodiment 76, wherein the apoptosis-inducing FasL moiety of (b) is wtFasL, mFasL, or sFasL.
E78. The pharmaceutical composition of embodiment 76, wherein the IL-2 moiety of (c) is wtIL-2, mIL-2, or sIL-2.
E79. The pharmaceutical composition of embodiment 76, wherein the chimeric protein of (b) binds through the FasL moiety to a further cell expressing a death receptor.
E80. The pharmaceutical composition of embodiment 76, wherein the chimeric IL-2 protein of (c) binds through the IL-2 moiety to a further cell expressing an IL-2 receptor.
E81. The pharmaceutical composition of embodiment 76, wherein the chimeric proteins of (b) and (c) forms tetramers and/or oligomers.
E82. The pharmaceutical composition of embodiment 76, wherein the member of a binding pair of (b) and (c) is streptavidin.
E83. The pharmaceutical composition of embodiment 82, wherein the streptavidin is core streptavidin.
E84. The pharmaceutical composition of embodiment 77 wherein the FasL of (b), is mFasL.
E85. The pharmaceutical composition of embodiment 78 wherein the IL-2 of (s), is mIL-2.
E86. The pharmaceutical composition of embodiment 76, wherein the apoptosis-inducing FasL moiety of (b) is positioned C-terminal to the binding pair member moiety.
E87. The pharmaceutical composition of embodiment 76, wherein the IL-2 moiety is positioned N-terminal to the binding pair member moiety.
E88. The pharmaceutical composition of embodiment 76, wherein said chimeric protein of (b) consists essentially of (i) an apoptosis-inducing FasL moiety and (ii) a member of a binding pair selected from the group consisting of avidin and streptavidin moieties.
E89. The pharmaceutical composition of embodiment 76, wherein said chimeric IL-2 protein consists essentially of (i) an IL-2 moiety and (ii) a member of a binding pair selected from the group consisting of avidin and streptavidin moieties.
E90. The pharmaceutical composition of embodiment 76, wherein the cell of (a) is a splenocyte.
E91. The pharmaceutical composition of embodiment 76, further comprising an alloantigen or autoantigen.
E92. The pharmaceutical composition of embodiment 91, wherein the alloantigen is a cell.
E93. The pharmaceutical composition of embodiment 91, wherein the alloantigen is a pancreatic islet cell, a tissue, or an organ.
E94. The pharmaceutical composition of embodiment 76, wherein the cell of (a) is selected from the group consisting of islet cells, bone marrow cells, hematopoietic stem cells, human beta cell products, hepatocytes, dendritic cells, mesenchymal cells, macrophages, endothelial cells, and T-cells.
E95. The pharmaceutical composition of embodiment 76, wherein the cell of (a) is part of a tissue or organ.
E96. A method of inducing immune modulation in a mammal in need thereof comprising administering an effective amount of the pharmaceutical composition of embodiment 76 to a mammal having a condition which is alleviated by the apoptosis of activated pathogenic lymphocytes.
E97. The method of embodiment 96, wherein the mammal has a condition selected from the group consisting of asthma, allergy, food poisoning, autoimmunity, and transplantation of allogeneic or xenogeneic tissue.
E98. The method of embodiment 97, wherein the autoimmune condition is selected from the group consisting of diabetes, multiple sclerosis, lupus erythematosis, sarcoidsis, and rheumatoid arthritis.
E99. The method of embodiment 96, further comprising administering an effective amount of a (a) chimeric protein comprising (i) an apoptosis-inducing FasL moiety and (ii) a member of a binding pair selected from the group consisting of avidin and streptavidin moieties, wherein the chimeric protein of is in soluble form and (b) soluble IL-2.
E100. The method of embodiment 99, further comprising administering an effective amount of rapamycin.
E101. The method of embodiment 100, further comprising administering an effective amount of cyclophosphamide.
E102. The method of embodiment 99, wherein the soluble IL-2 is a chimeric protein comprising (i) an IL-2 moiety and (ii) a member of a binding pair selected from the group consisting of avidin and streptavidin moieties.

### EXAMPLES

The following examples are given by way of illustration and are in no way intended to limit the scope of the present invention.

### Example 1 Construction, Expression, Characterization, and Cell Surface Decoration of SA-FasL and SA-IL-2

Construction of SA-FasL and SA-IL-2 and expression in Drosophila S2 cells. Genomic DNA was isolated from Streptomyces avidinii (ATCC Cat. #27419) and 0.2 μg of this DNA was used as template for amplification using primers specific for the 5′-end and 3′-end of core streptavidin in PCR. The 5′-primer included sequences for BglII and 6 His residues (SEQ ID NO: 11) to allow cloning in frame with the Drosophila secretion signal (BiP) for expression as a secreted protein and purification using Ni-affinity columns. (Alternatively, Flag tag can be used instead of the 6 His residues (SEQ ID NO: 11), and anti-Flag Abs can be used for purification.) The PCR product was cloned into the TA cloning vector (Invitrogen, San Diego, CA) and several positive clones were identified by sequencing. We next subcloned the extracellular domain of rat FasL without the metalloproteinase site using a wild type FasL cDNA clone as a template and a sense primer to the 5′-end of the extracellular region, FasL6 (nucleotides 428-453) containing an EcoRI site, and an antisense primer to the 3′-end untranslated region of FasL, FasL2 (nucleotides 977-998) containing an EcoRI site in frame with SA, in PCR. Both the core streptavidin and extracellular FasL clones were digested out of the TA cloning vector with BglII-EcoRI for SA and EcoRI for extracellular FasL. These DNA inserts were then subcloned into the BglII-EcoRI-cut pMT/BiP/V5-His vector for expression in the DES™ system (Invitrogen).

Drosophila S2 cells were transfected with 20 μg of pMT/BiP/V5-His expression vector containing the SA-FasL recombinant gene in frame with the BiP secretion signal using the Calcium Phosphate Transfection kit according to the manufacturer's instructions (Invitrogen). Stable transfectants were established by cotransfection with 1 μg of pCoHYGRO vector and maintained in the presence of 300 μg/ml of hygromycin. SA-FasL expression was induced with 600 μM copper sulfate. Supernatant was collected 1-4 days after induction and either used immediately or precipitated with 50% ammonium persulfate, dialyzed against PBS, and purified using Ni-NTA columns (QIAGEN, Valencia, CA). The concentration of purified or culture supernatant SA-FasL was determined by the Bradford method or ELISA, respectively, using known amounts of commercially available streptavidin as standard. Similar methods can be used for the construction, expression, and purification of SA-IL-2.

Characterization of SA-FasL by Western blot and ELISA. The expression of SA-FasL by S2 cells was first detected using biotin-coated microwell strip plates in ELISA (Pierce, Rockford, IL). Briefly, biotinylated wells were incubated with culture supernatants collected after 96 hrs of induction of S2 cells with CuSO₄ for 45 min at room temperature. Wells were washed extensively, incubated with the working concentrations of primary antibodies against streptavidin or FasL (MFL4) for 45 min. Alkaline phosphatase-or HRP-conjugated secondary antibodies were used with the appropriate substrates to assess the amount of chimeric protein using an ELISA reader (Victor, Wallac, Gaithersburg, MD).

For Western blot analysis, culture supernatants were fractionated by PAGE under native and denaturing conditions and transferred onto PVDF membranes using a dry-blot apparatus (BioRad). Membranes were incubated with blocking buffer (5% dry milk and 0.5% Tween 20 in PBS), followed by incubation with rabbit anti-rat FasL serum (C-178; Santa Cruz, CA) at 1:1000 dilution in the blocking buffer for 1 hr. Membranes were then washed 3 times with washing buffer (0.2% Tween 20 in PBS) and incubated 1 hr with HRP-conjugated goat anti-rabbit antibody at 1:5000 dilution in blocking buffer (Pierce, Rockford, IL). Finally, membranes were washed several times and incubated in a chemiluminescent substrate according to the manufacturer's instructions (Molecular Dynamics, San Diego, CA).

Modification of the cell membrane with biotin and decoration with SA-FasL. Various cell types were incubated in 1.5-150 μM freshly prepared EZ-Link™ Sulfo-NHS-LC-Biotin (Pierce) in PBS for 30 min at room temperature. Cells were washed twice and resuspended in PBS supplemented with 50-100 ng of SA-FasL per 10⁶ cells. After incubation on ice for 20-30 min with intermittent mixing, cells were washed twice and analyzed in flow cytometry using streptavidin-APC and MFL4 labeled with different fluorochromes to assess the cell-surface levels of biotin and SA-FasL, respectively. Similar methods were used to modify the cell membrane of cells with SA-IL-2.

### Example 2 SA-FasL as a Soluble Protein for Immunomodulation

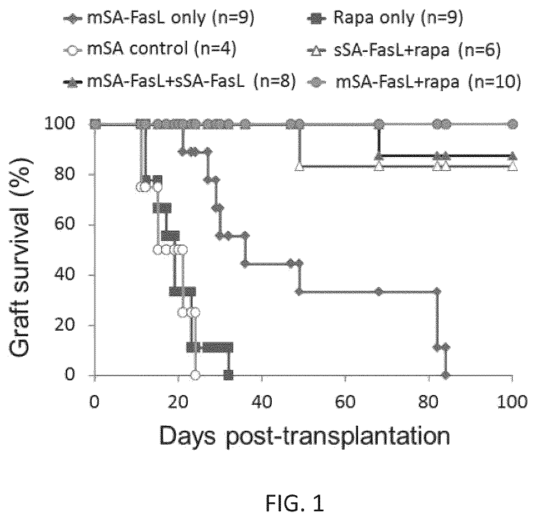

Immunomodulation with SA-FasL-engineered islets requires a short course of rapamycin treatment for tolerogenic efficacy (FIG. 1). This may be due to the limited amount of SA-FasL protein displayed on islets or short duration because of high turnover kinetics, thereby minimizing its tolerogenic effect. We, therefore, asked if SA-FasL can be used as a soluble protein to obviate the need for rapamycin. The use of SA-FasL as a soluble protein over the form attached to biological membranes for immunomodulation has various advantageous. First, the clinical and regulatory paths for using immunomodulatory biologics are well established. Second, the use of soluble biologics for therapy is practical as it involves simple injections. Third, the dose and treatment frequency and duration of soluble biologics can easily be established for the desired therapeutic outcome. Importantly, SA-FasL in soluble form may have wider applications as compared with the form fixed on biological surfaces.

Intraperitoneal treatment of graft recipients with 2-5 μg SA-FasL protein/mouse on days 1, 3, 5, 7 post-transplantation resulted in robust tolerogenic efficacy to allogeneic SA-FasL-islet grafts as only ⅛ had delayed rejection (˜70 days; FIG. 1). Treatment with soluble SA-FasL only under a short course of rapamycin was also effective, achieving >80% graft survival.

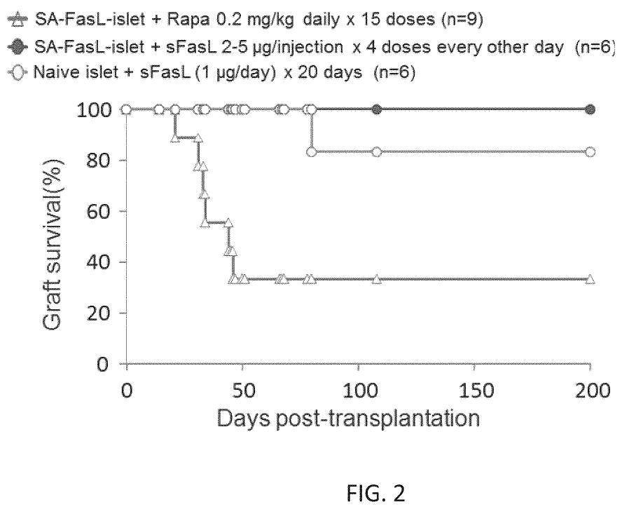

To further establish SA-FasL as a soluble biologic, we tested its immunomodulatory capacity for the prevention of allogeneic islets transplanted using a more stringent model of intraportal islet transplantation. The rationale for this model is twofold. First, intraportal islet transplantation is the only site practiced in the clinic. Second, intraportal islet transplantation provides a much more stringent model as compared to subrenal islet transplantation. As shown in FIG. 2, only 30% of recipients receiving allogeneic islets engineered with SA-FasL have long-term survival when transplanted intraportally in the BALB/c-to-C57BL/6 model. This is to be contrasted to 100% survival when SA-FasL-engineered islets transplanted under the kidney capsule. Therefore, we used this model to test if SA-FasL can be developed as a soluble biologic for the prevention and treatment of type I diabetes and foreign graft rejection. All BALB/c islets engineered with SA-FasL protein survived when 4 doses of SA-FasL (2-5 μg/injection) were administered into C57BL/6 recipients intravenously or intraperitoneally on days 1, 3, 5, and 7 post-transplantation. Indeed, the need for islet engineering with SA-FasL can also be overcome by systemic and protracted use of soluble FasL (1 μg injection daily for 20 days starting on day of transplantation). Only one out of six mice rejected the graft on day 80 post-transplantation.

These observations are important from two perspectives; i) for demonstrating that SA-FasL as monotherapy is sufficient to induce tolerance, but that the window of immunomodulation needs to be extended, and ii) SA-FasL can be used as a soluble biologic for immunomodulation at therapeutic doses without toxicity.

### Example 3 SA-FasL as an Effective Immunomodulator to Control T Memory Responses

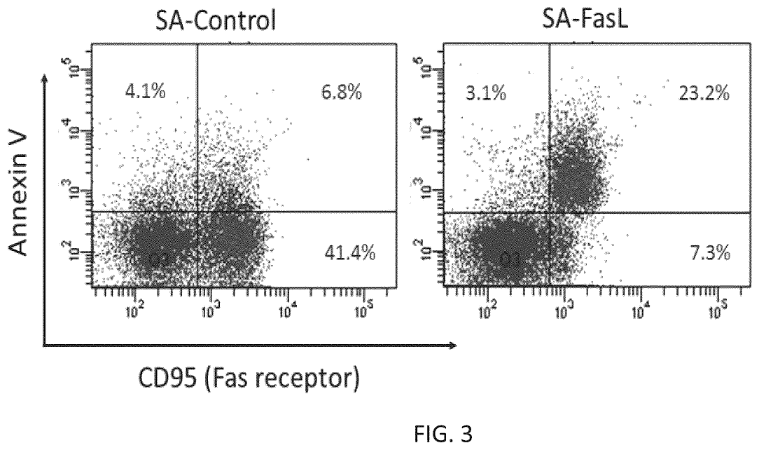

Inasmuch as individuals with onset of autoimmunity have a large pool of activated and memory T cells, we asked if SA-FasL is capable of inducing apoptosis in such cells. For this purpose, we used nonhuman primate (NHP) peripheral blood lymphocytes (PBMCs). The PBMCs from 3 different NHPs were incubated with SA-FasL protein and apoptosis was assessed using flow cytometry. Almost all T cells expressing Fas underwent apoptosis irrespective of their phenotype, Teff (CD3⁺CD28⁻Fas⁺) or T central (CD3⁺CD28⁺Fas⁺) memory (FIG. 3).

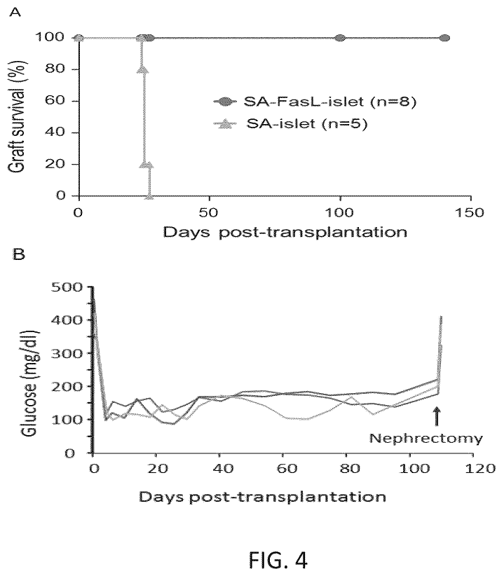

To further provide in vivo data for efficacy of SA-FasL to overcome rejection in recipients with a large T cell memory pool, a set of C57BL/6 mice were transplanted with BALB/c skin grafts. These mice rejected skin grafts within 20 days. Mice were then made diabetic using STZ and transplanted with BALB/c SA-FasL-islet grafts 40-50 days post skin transplantation under transient cover of rapamycin. All mice remained euglycemia for an observation period over 140 days (FIG. 4A). Surgical removal of the kidney harboring islets resulted in prompt hyperglycemia, demonstrating that euglycemia was due to islet grafts (FIG. 4B). Taken together, these observations are important as various tolerance protocols fail to eliminate/control T memory cells, which serve as a major barrier for achieving clinical tolerance.

### Example 4 SA-FasL as an Immunomodulator to Prevent Type 1 Diabetes

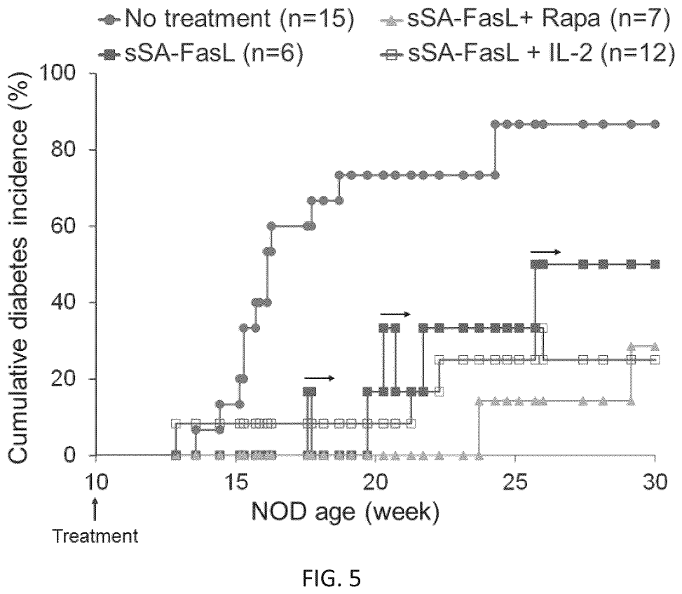

Inasmuch as antigen-experienced cells express Fas receptor on their surface, systemic treatment with SA-FasL has potential to eliminate such cells and control/reverse T1D without a major effect on quiescent T cell repertoire. This notion was tested by treating 10-wk female NOD mice daily with 500 ng SA-FasL protein for 30 days. As shown in FIG. 5, this treatment significantly delayed the onset and incidence of T1D as compared with controls. We did not observe any sign of SA-FasL toxicity. We next asked the question if SA-FasL will work in synergy with IL-2. Without being bound by theory, we hypothesized that IL-2 can further enhance FasL mediated apoptosis of T cells by various means, including down-regulating anti-apoptotic genes, such as cFLIP. In marked contrast, IL-2 is also critical growth and survival factor for T regulatory cells and T regulatory cells are resistant to Fas-mediated apoptosis due to the expression of high levels of anti-apoptotic genes, such as cFLIP. Indeed, T regulatory cells express FasL on their surface and control T effector responses by direct elimination via Fas receptor or indirectly by eliminating Fas-expressing DCs. Therefore, we hypothesized that the combinatorial use of SA-FasL and IL-2 may be effective in immune modulating auto and alloreactive responses for therapeutic purposes.

Treatment of female NOD (10-wk old) with a combination of SA-FasL and 3000 IU IL-2 daily for 30 days resulted in much improved delay of onset and incidence of diabetes. Indeed, the SA-FasL+IL-2 regimen performed as well as SA-FasL+rapamycin with respect to prevention of diabetes. These data demonstrate that SA-FasL is effective in delaying the onset and preventing the incidence of type 1 diabetes in NOD and works in synergy with IL-2 or rapamycin for a better outcome.

### Example 5 SA-FasL Synergized With IL-2 to Promote Long-Term Survival of Transplanted Pancreatic Islets

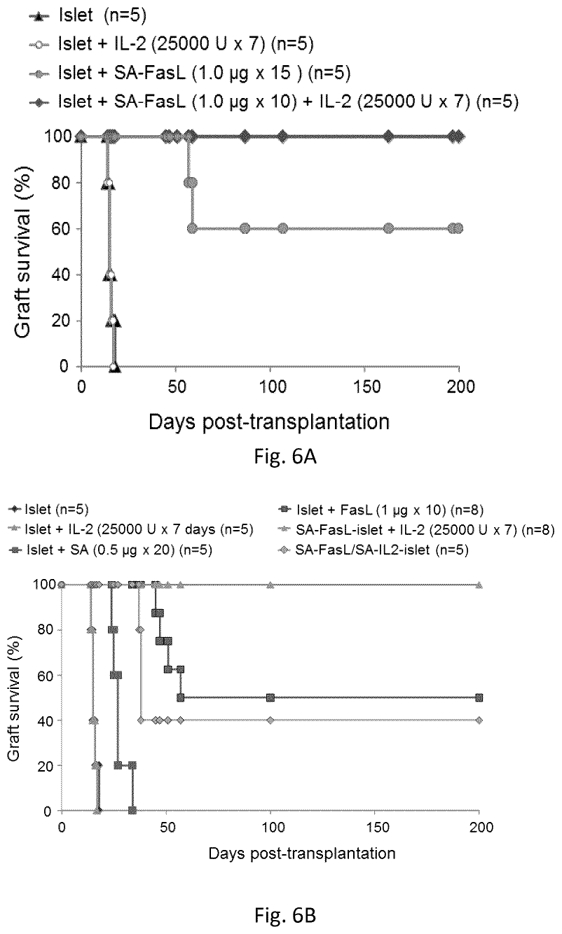

The combined use of soluble forms of SA-FasL and IL-2 in modulating immune response to autoantigens and preventing diabetes in NOD led us to test the combined use of these molecules as soluble biologics and a combination of soluble biologics and displayed on the surface of pancreatic islets. C57BL/6 mice were treated with intravenous injection of streptozotocin (200 mg/kg) and diabetes was confirmed by two consecutive blood glucose readings higher than 300 mg/dl. Pancreatic islets were harvested from BALB/c mice, cultured overnight, and then either left unmodified or engineered with SA-FasL alone or in combination with SA-IL2. These islets were then immediately transplanted intraportally into diabetic mice (450-550 islets/mouse). Mice were then treated with the indicated proteins and doses. Animals were monitored for diabetes, and those with two consecutive daily measurements of ≥250 mg/dl blood glucose level were considered diabetic and confirmation of graft failure. The data demonstrate that soluble SA-FasL as monotherapy prevents the rejection of ˜60% of allogeneic islets and soluble IL-2 work in synergy and prevents the rejection in 100% of recipients long-term (FIG. 6A). Soluble IL-2 also works in synergy with SA-FasL on the surface of islet to prevent graft rejection in all graft recipients. Importantly, co-display of both SA-FasL and SA-IL2 was also effective in preventing rejection of ˜40% of grafts (FIG. 6B).

### Example 6 SA-FasL Protein as a Biologic for the Prevention of Cardiac Allograft Rejection

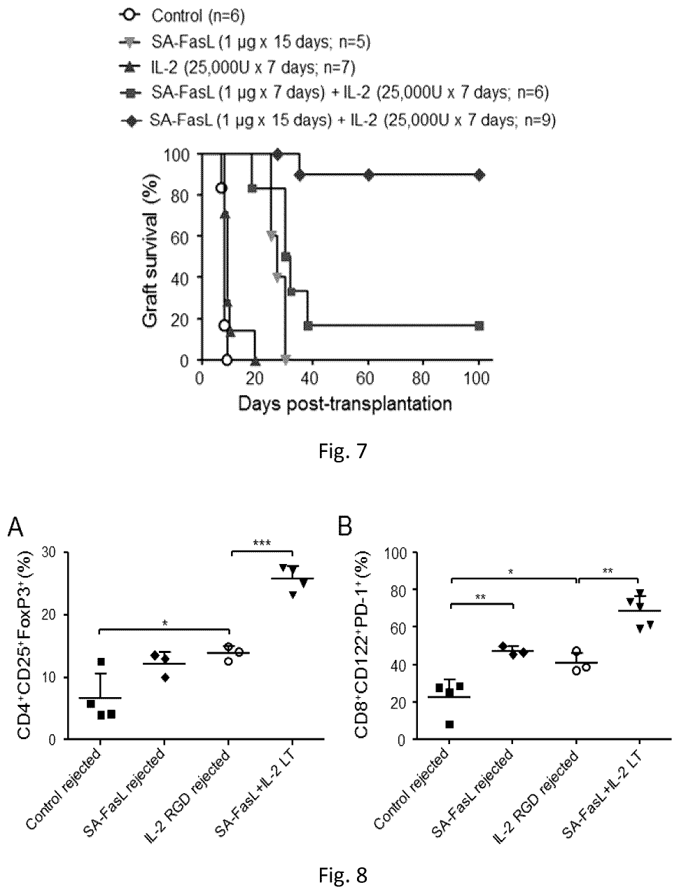

We next tested if SA-FasL as a biologic can work as an immunomodulator to prevent rejection of vascularized solid grafts. Heart transplantation from BALB/c into C57BL/6 mice was used as a stringent allogeneic heart graft model. C57BL/6 recipients of allogeneic BALB/c hearts were treated with a short course of IL-2 (7 doses) and SA-FasL (15 doses) without any other manipulation. As shown in FIG. 7, SA-FasL as monotherapy caused moderate prolongation of the grafts, while IL-2 did not have a significant effect. However, the combination of both molecules had robust tolerance effects with only 1 out of 9 grafts showing delayed rejection, whereas the rest survived for a 100-day observation period (data is from >3 independent experiments and hearts were scored in blind fashion by 2 individuals). We also demonstrated that soluble use of SA-FasL in combination with rapamycin was also effective in preventing cardiac graft rejection (Table 1).

TABLE 1
SA-FasL works in synergy with rapamycin to prevent allograft rejection.

| | | | | Rapamycin | | |
|---|---|---|---|---|---|---|
| Grp | Donor | Recipient | Soluble proteins | (mg/kg) | N | Graft Survival |
| 1 | BALB/c | C57BL/6 | None | None | 4 | 7, 8, 8, 8 |
| 2 | BALB/c | C57BL/6 | None | 0.2 daily for 15 | 4 | 17, 20, 20, 22 |
| | | | | doses | | |
| 4 | BALB/c | C57BL/6 | SA-FasL 1 μg, iv, | 0.2 daily for 15 | 6 | >70, >70, >100, |
| | | | daily for 15 days | doses | | >100, >100, >100 |

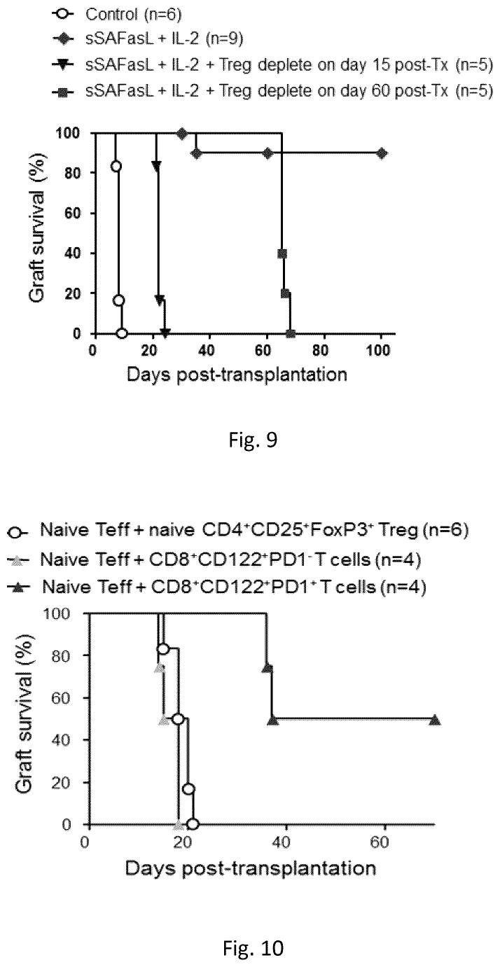

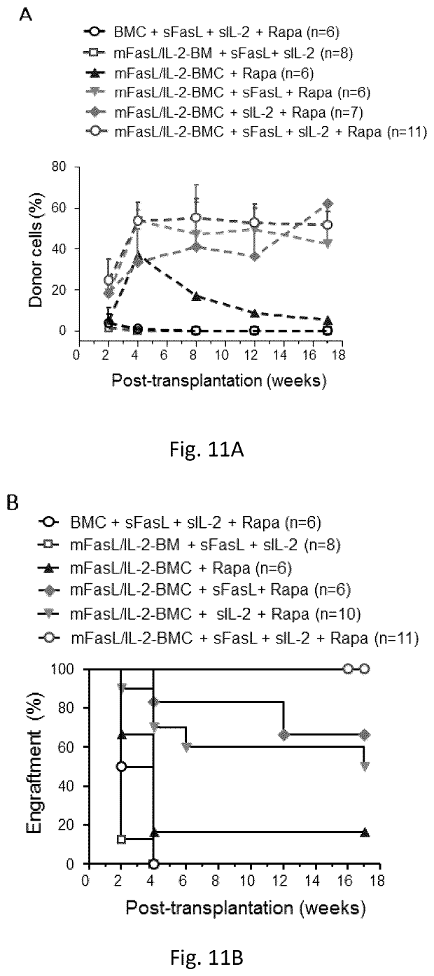

Mechanistic studies demonstrated a higher frequency of both CD4⁺CD25⁺FoxP3⁺ T reg as well as CD8⁺CD122⁺PD-1⁺ T cells in graft draining lymph node (not shown) and heart graft (FIG. 8). Depletion of CD4⁺CD25⁺FoxP3⁺ T reg cells 15 or 60 days after transplantation negated the tolerogenic effect of SA-FasL and IL-2 and resulted in prompt graft rejection (FIG. 9). CD8⁺CD122⁺PD-1⁺ T cells had regulatory function as i) depletion of these cells in long-term mice (>100 days) resulted in graft rejection in ˜50% of recipients, and ii) flow cytometry sorted CD8⁺CD122⁺PD-1⁺, but not CD8⁺CD122⁺PD-1⁻, T cells prevented rejection of BALB/c hearts in an adoptive transfer model using Rag KO mice (FIG. 10).

The data shows that SA-FasL and IL-2 is surprisingly effective at inducing long term tolerance. The use of IL-2 by itself has no effect on inducing tolerance, but IL-2 synergistically enhances the effect of FasL in inducing long term tolerance to selected antigens. Therefore, the combined use of SA-FasL and IL-2 constitutes a major improvement in treating autoimmune disease and in inducing transplantation tolerance.

### Example 7 SA-FasL Protein Work in Synergy With IL-2 to Prevent Bone Marrow Graft Rejection and Establish Mixed Hematopoietic Chimerism

Bone marrow transplantation (BMT) as a source of hematopoietic stem cells is perceived as a powerful therapeutic regimen that can potentially treat a variety of inherited and acquired diseases, such as enzyme deficiencies, metabolic disorders, cancer. BMT leading to mixed allogeneic hematopoietic chimerism (MAHC) can be used as an immunomodulatory approach to induce tolerance to auto-, allo-, and xenoantigens for the treatment of autoimmune disorders and prevention of foreign graft rejection. Mixed chimeras are tolerant to both donor and host antigens since i) stem cells in the donor bone marrow give rise to immune cells, such as lymphocytes, that are “educated” in the host immune environment for nonresponsiveness to the host antigens and ii) macrophages and dendritic cells arising from the donor bone marrow serve as antigen-presenting cells in the thymus to eliminate donor reactive host lymphocytes. In addition to this “central tolerance”, other peripheral immunoregulatory mechanisms, such as clonal anergy and immune suppression, appear to contribute to the overall tolerance observed in mixed hematopoietic chimeras.

Establishment of MAHC using BMT suffers from three major obstacles; graft-versus-host (GVH) reaction, host-versus-graft (HVG) reaction, and stem cell competition. GVH reactions are primarily mediated by mature donor T cells in the transplanted bone marrow inoculum that recognize host alloantigens and mount an immune response, resulting in graft-versus-host disease (GVHD). GVHD affects many organs and often yields life threatening complications. Although depletion of mature T cells from BM grafts is effective in preventing GVHD, it is associated with a high incidence of engraftment failure. Current treatment for GVHD involves nonspecific immunosuppression using various agents. However, although somewhat effective in preventing GVHD, the chronic use of these agents is associated with increase incidences of secondary malignancies and infections.

Rejection of allogeneic BMC by the host mature T cells represents a second hurdle that needs to be overcome in order to establish mixed chimerism. A series of approaches have been implemented to physically and functionally eliminate host T cells reactive to donor BMC for the establishment of mixed chimerism. These regimens mostly rely on nonspecific immunosuppressive regimens or generalized lymphocyte-specific interventions. All of these approaches can potentially render the host immunoincompetent. Therefore, it is important to develop clinically applicable protocols that are more effective, specific for the elimination of pathogenic T cells, and involves minimal conditioning of the host.

The third important barrier for the establishment of mixed hematopoietic chimerism is the competition between the donor and host HSC for available niches. In order to facilitate engraftment, most mixed chimerism protocols developed to date rely on host preconditioning to achieve some level of host HSC depletion. Total body irradiation has been the method of choice because of its reproducibility and case of administration.

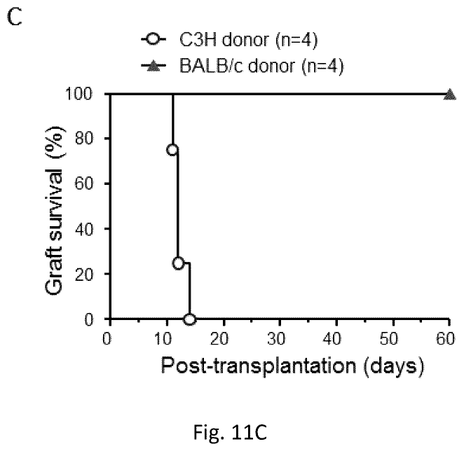

The efficacy of combined use of soluble SA-FasL and IL-2 in inducing tolerance to allogeneic pancreatic islets as tissue graft led us to test if this regimen is also effective in inducing tolerance to a solid organ. C57BL/6 mice were subjected to 300 cGy total body irradiation followed by intravenous transplantation of 30×10⁶ unmodified (BMC) or SA-FasL (mFasL-BMC) or the combination of SA-FasL and SA-IL-2 (mFasL/IL-2-BMC) one day later. FIG. 11A-C. Selected groups of mice were also treated i.p. with 500 ng/daily for 15 doses of soluble SA-FasL (sFasL), 25,000 IU/daily for 7 doses of soluble IL-2 (sIL-2; human IL-2 obtained commercially or produced in house using insect cells transfected with human IL-2 cDNA engineered to include a 6×His tag (SEQ ID NO: 11) for purification) or combination of both molecules (sFasL+sIL-2). The indicated groups also were treated i.p. with 10 daily doses of rapamycin at 0.5 mg/kg. Percent donor chimerism at various times post-transplantation was assessed using peripheral blood lymphocytes with antibodies against donor class I MHC molecule in flow cytometry. FIG. 11A, average donor chimerism, FIG. 11B, percent engraftment, FIG. 11C, Long-term chimeric mice reject C3H third-party, but not BALB/c donor skin, demonstrating donor-specific tolerance.

These data demonstrated that the generation of durable mixed allogeneic chimerism in mice pretreated with 300 cGy total body irradiation required both SA-FasL/IL-2 attached to the surface of BM cells as well as systemic treatment of recipients either with soluble SA-FasL or IL-2. However, the systemic use of both molecules as biologics improved the efficacy of percent donor chimerism as well as engraftment because all the recipients were successfully engrafted. Importantly, long-term chimeric mice were immunocompetent and tolerant to donor antigens as they rejected third party, but not donor, skin grafts. Therefore, the combined use of SA-FasL and IL-2 improves long-term transplant tolerance.

### Example 8 Cyclophosphamide Worked in Synergy With the SA-FasL and IL-2 Combination to Establish Mixed Hematopoietic Allogeneic Chimerism

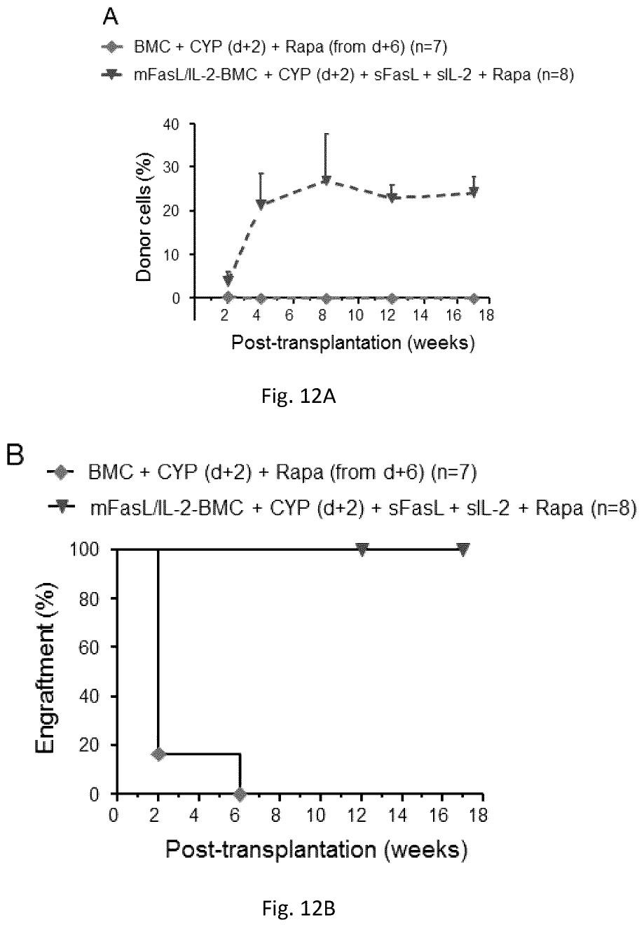

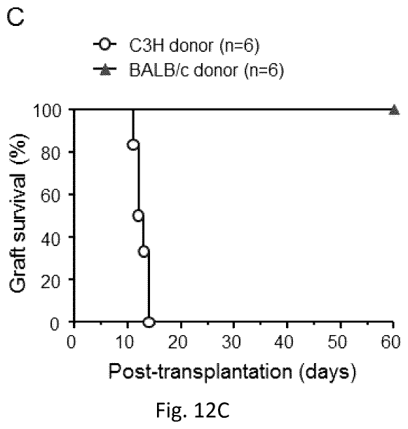

We further tested whether other immunomodulatory agents such as cyclophosphamide can further improve the effect of SA-FasL and IL-2 in establishing mixed hematopoietic allogeneic chimerism when low dose of total body irradiation is used. C57BL/6 mice were subjected to 100 cGy total body irradiation followed by intravenous transplantation of 30×10⁶ unmodified (BMC) or the combination of SA-FasL and SA-IL-2 (mFasL/IL-2-BMC) one day later. Selected groups of mice were also treated i.p. with cyclophosphamide (200 mg/kg) on day+2. All groups were treated i.p. with rapamycin (2 mg/kg) daily for 10 doses starting on day 6 pot-transplantation. The test group was also treated starting on day 6 post-transplantation with both soluble SA-FasL (sFasL; 500 ng/daily for 15 doses) and IL-2 (25,000 IU/daily for 7 doses). Percent donor chimerism at various times post-transplantation was assessed using peripheral blood lymphocytes with antibodies against donor class I MHC molecule in flow cytometry. FIG. 12A, average donor chimerism, FIG. 12B, percent engraftment, FIG. 12C, long-term chimeric mice reject C3H third-party, but not BALB/c donor skin, demonstrating donor-specific tolerance. Nonchimeric control mice (BMC+cyclophosphamide+rapamycin) rejected grafts from both donor and third party (data not shown).

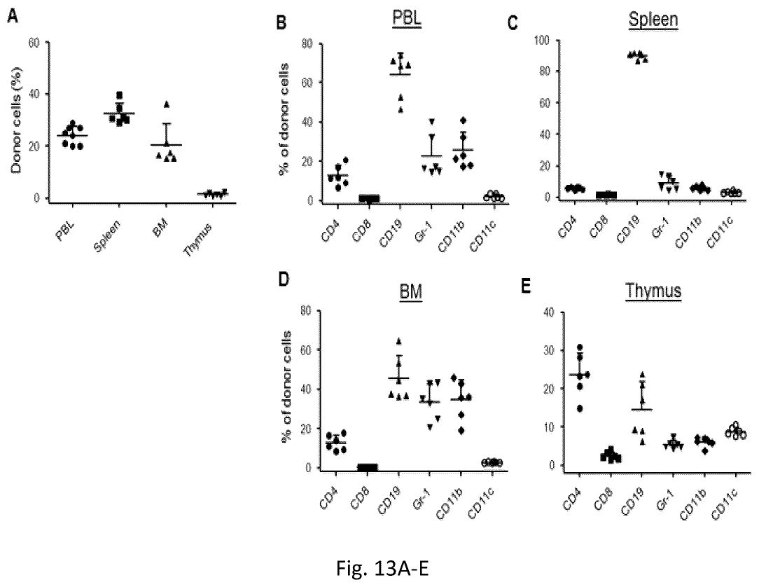

These data demonstrated that cyclophosphamide acted synergistically in combination with SA-FasL and IL-2 to establish mixed hematopoietic allogeneic chimerism. Furthermore, analysis of various tissues from mice with durable donor mixed chimerism revealed that the treatment induced long-term multilineage donor chimerism (FIG. 13A-B).

### Example 9 Mice With Durable Donor Mixed Chimerism Generate In Vitro Response to Third Party, but not Donor Alloantigens

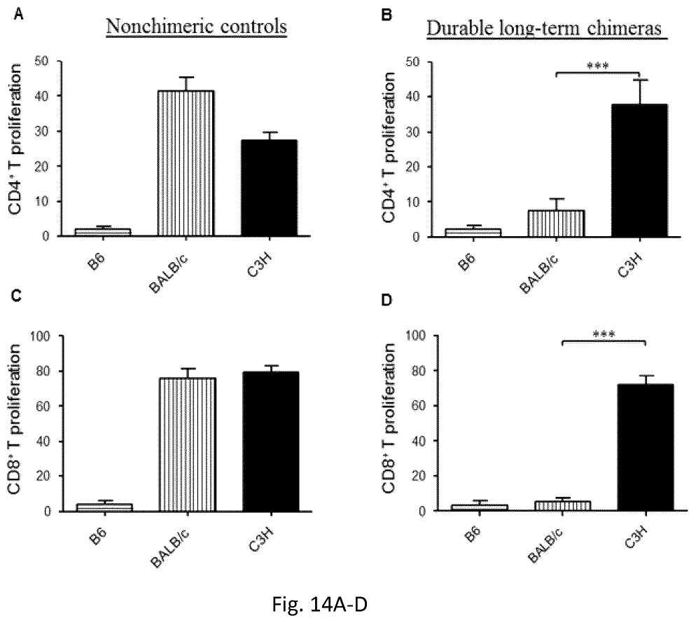

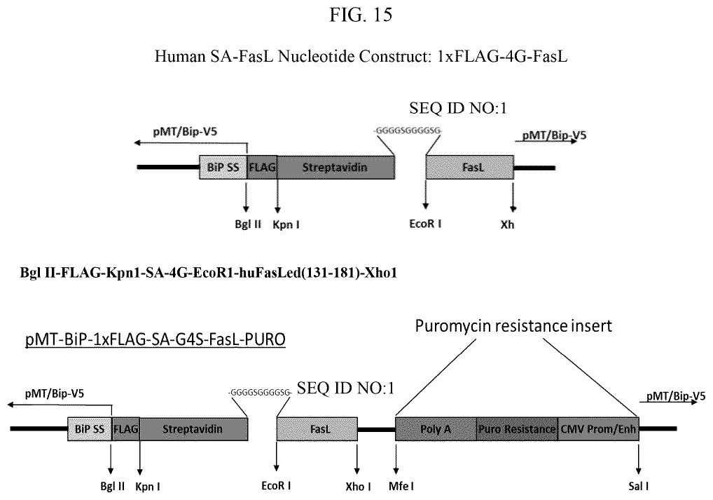

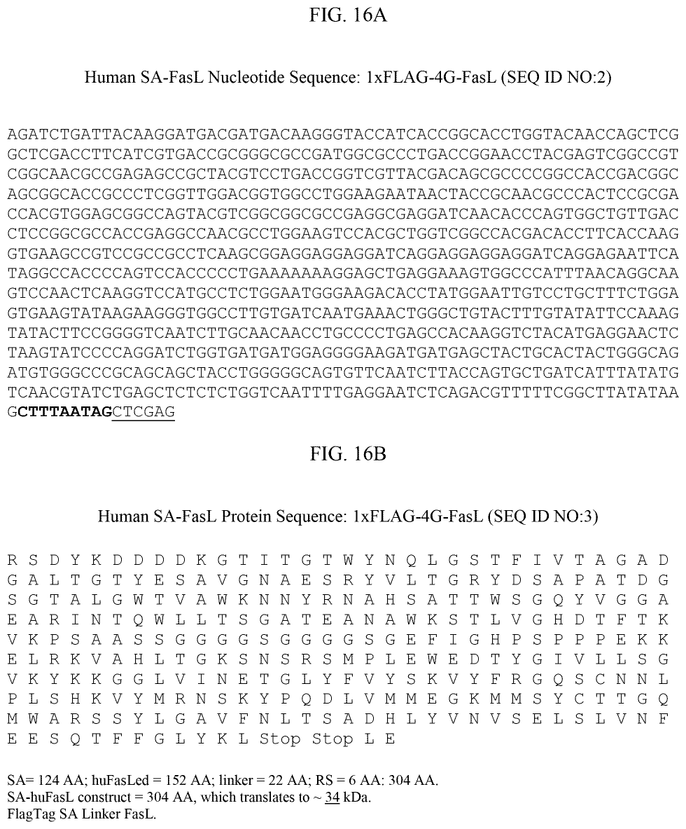

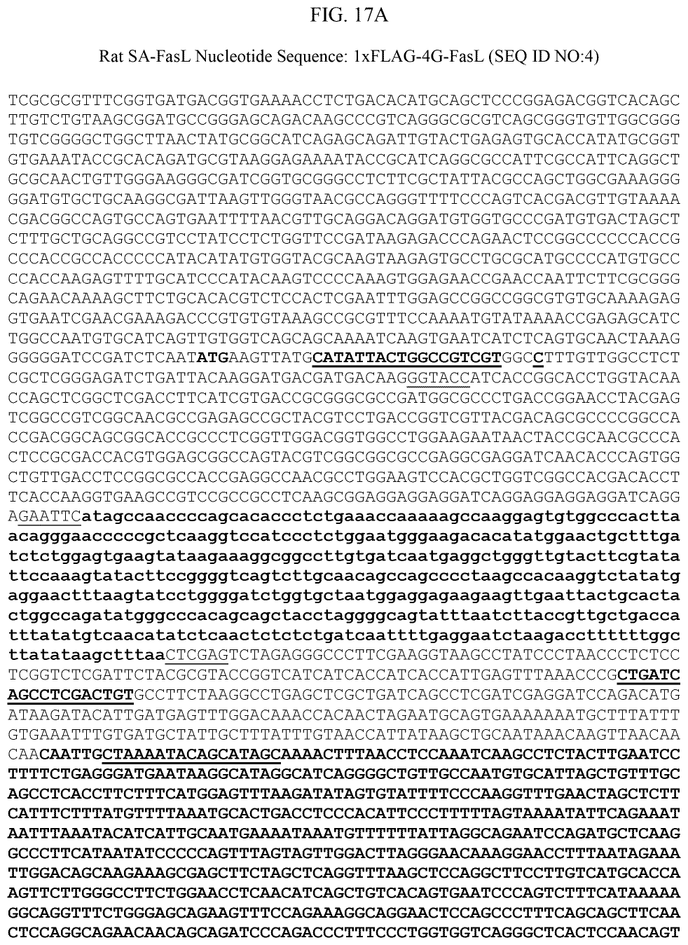

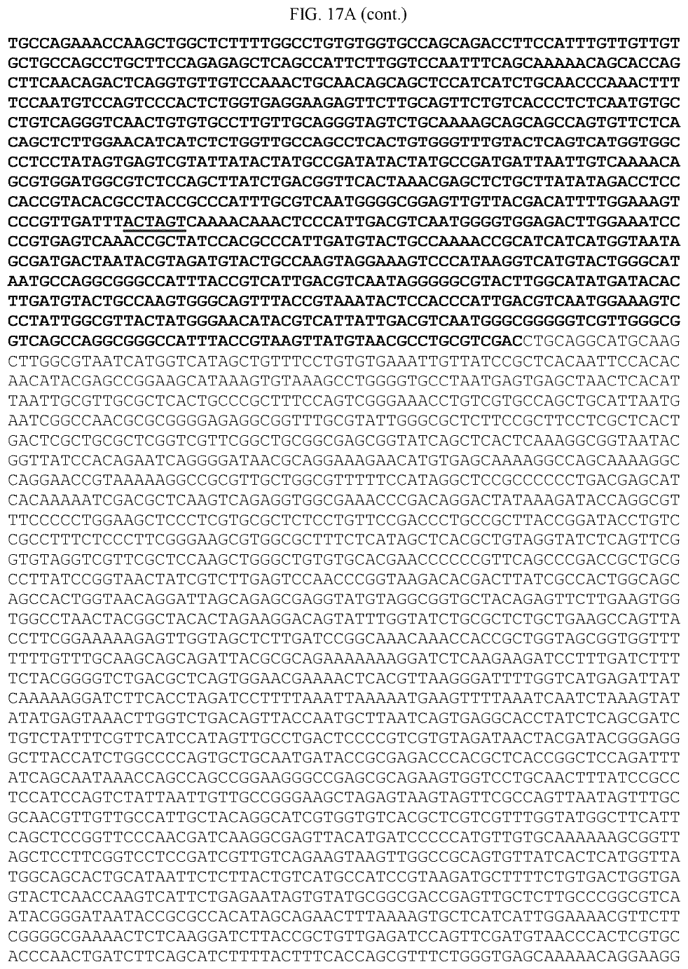

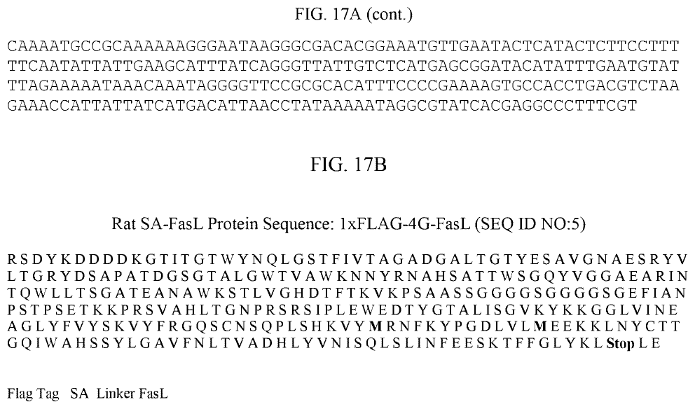

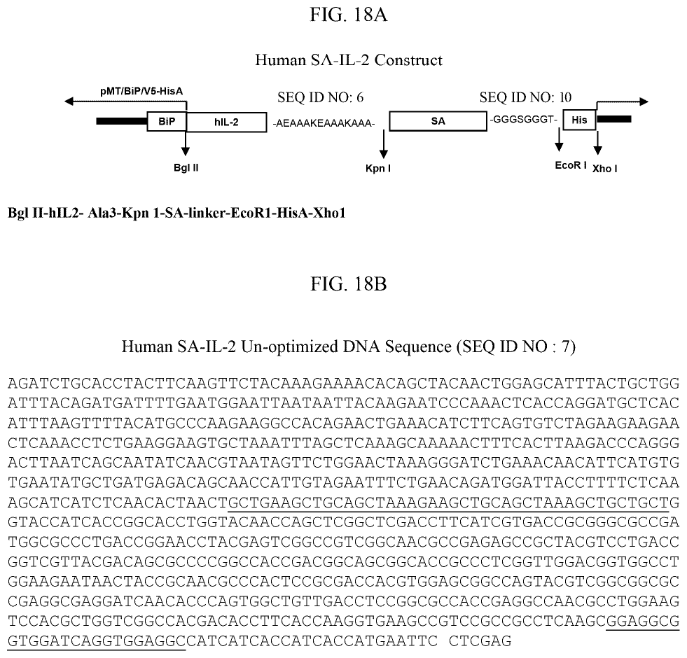

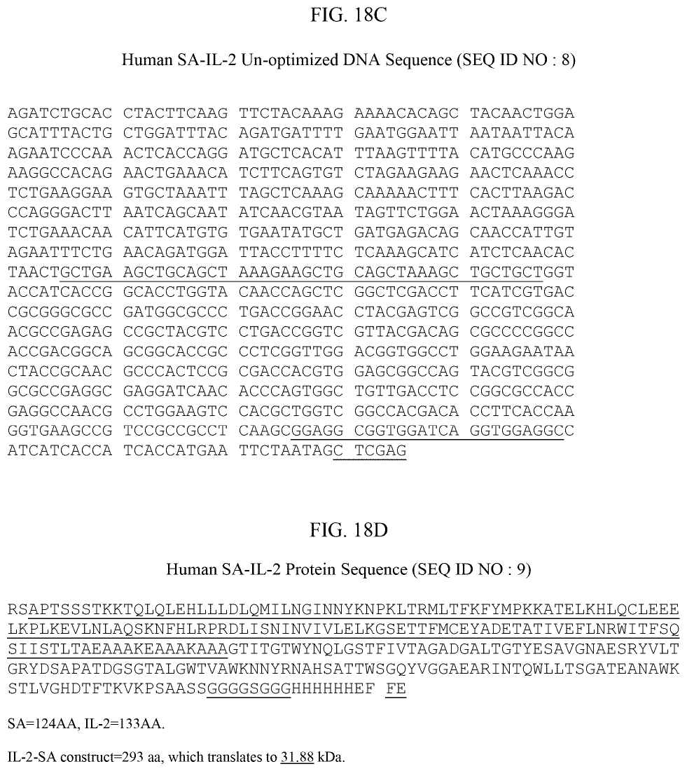

To further test the immunocompetence of mice having established durable donor mixed chimerism after transplantation, spleens were harvested from long-term (>100 days) durable mixed chimeras and control mice that rejected donor BMCs shown in FIG. 12A-B. Spleens were processed into single cell suspension, labeled with 2.5 μM CFSE, resuspended in DMEM, and 50×10⁶ CFSE labeled splenocytes were plated on a petri dish for 45 minutes at 37° C. to enrich lymphocytes. After 45 minutes non-adherent cells were collected, washed, and incubated (1×10⁵ cells) with irradiated (2000 cGy; 1×10⁵ cells) donor BALB/c or C3H splenocytes as stimulators in 96-well U-bottom titer plates in mixed lymphocyte reaction medium. Irradiated syngeneic C57BL/6 cells were used as controls. After 4 days, cells were stained with fluorochrome-labeled antibodies against rat CD4 and CD8, analyzed by flow cytometry gating on live cells, and the percentage of proliferating cells is graphed. As demonstrated by the data in FIG. 14, long-term chimeric animals generated a proliferative response to C3H third party stimulators, but not BALB/c donor antigens, demonstrating immune competence and antigen-specific tolerance. In marked contrast, lymphocytes from nonchimeric mice generated a proliferative response to both C3H third party as well as BALB/c stimulators, demonstrating lack of antigen-specific tolerance.

1-17. (canceled)

18. A method for inducing immune tolerance in a subject in need thereof, comprising administering to the subject a soluble chimeric FasL protein comprising a FasL moiety and a streptavidin or avidin moiety.

19-23. (canceled)

24. The method of claim 18, wherein the soluble chimeric FasL protein has the amino acid sequence of SEQ ID NO: 3.

25. (canceled)

26. The method of claim 18, wherein the soluble chimeric FasL protein is administered by injection.

27. The method of claim 18, wherein the soluble chimeric FasL protein is administered by intravenous or intraperitoneal injection.

28. The method of claim 18, wherein the subject is in need of treatment for type 1 diabetes.

29. The method of claim 18, wherein the subject is in need of treatment or prevention of allograft rejection.

30. The method of claim 18, wherein the method further comprises administering rapamycin to the patient.


SEQUENCE LISTING (11 sequences)

SEQ ID NO: 1 - protein, 11 aa, synthetic construct
  REGION 1..11: note = Description of Artificial Sequence: Synthetic peptide
GGGGSGGGGSG

SEQ ID NO: 2 - DNA, 912 bp, synthetic construct
  misc_feature 1..912: note = Description of Artificial Sequence: Synthetic polynucleotide
  source 1..912: mol_type = other DNA
agatctgattacaaggatgacgatgacaagggtaccatcaccggcacctggtacaaccagctcggctcgaccttcatcgtgaccgcgggcgccgatggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgttgacctccggcgccaccgaggccaacgcctggaagtccacgctggtcggccacgacaccttcaccaaggtgaagccgtccgccgcctcaagcggaggaggaggatcaggaggaggaggatcaggagaattcataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctttaatagctcgag

SEQ ID NO: 3 - protein, 300 aa, synthetic construct
  REGION 1..300: note = Description of Artificial Sequence: Synthetic polypeptide
RSDYKDDDDKGTITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSGGGGSGGGGSGEFIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 4 - DNA, 6012 bp, synthetic construct
  misc_feature 1..6012: note = Description of Artificial Sequence: Synthetic polynucleotide
  source 1..6012: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccagtgaattttaacgttgcaggacaggatgtggtgcccgatgtgactagctctttgctgcaggccgtcctatcctctggttccgataagagacccagaactccggccccccaccgcccaccgccacccccatacatatgtggtacgcaagtaagagtgcctgcgcatgccccatgtgccccaccaagagttttgcatcccatacaagtccccaaagtggagaaccgaaccaattcttcgcgggcagaacaaaagcttctgcacacgtctccactcgaatttggagccggccggcgtgtgcaaaagaggtgaatcgaacgaaagacccgtgtgtaaagccgcgtttccaaaatgtataaaaccgagagcatctggccaatgtgcatcagttgtggtcagcagcaaaatcaagtgaatcatctcagtgcaactaaaggggggatccgatctcaatatgaagttatgcatattactggccgtcgtggcctttgttggcctctcgctcgggagatctgattacaaggatgacgatgacaagggtaccatcaccggcacctggtacaaccagctcggctcgaccttcatcgtgaccgcgggcgccgatggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgttgacctccggcgccaccgaggccaacgcctggaagtccacgctggtcggccacgacaccttcaccaaggtgaagccgtccgccgcctcaagcggaggaggaggatcaggaggaggaggatcaggagaattcatagccaaccccagcacaccctctgaaaccaaaaagccaaggagtgtggcccacttaacagggaacccccgctcaaggtccatccctctggaatgggaagacacatatggaactgctttgatctctggagtgaagtataagaaaggcggccttgtgatcaatgaggctgggttgtacttcgtatattccaaagtatacttccggggtcagtcttgcaacagccagcccctaagccacaaggtctatatgaggaactttaagtatcctggggatctggtgctaatggaggagaagaagttgaattactgcactactggccagatatgggcccacagcagctacctaggggcagtatttaatcttaccgttgctgaccatttatatgtcaacatatctcaactctctctgatcaattttgaggaatctaagaccttttttggcttatataagctttaactcgagtctagagggcccttcgaaggtaagcctatccctaaccctctcctcggtctcgattctacgcgtaccggtcatcatcaccatcaccattgagtttaaacccgctgatcagcctcgactgtgccttctaaggcctgagctcgctgatcagcctcgatcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctcaggtttaagctccaggcttccttgtcatgcaccaagttcttgggccttctggaacctcaacatcagctgtcacagtgaatcccagtctttcataaaaaggcaggtttctgggagcagaagtttccagaaaggcaggaactccagccctttcagcagcttcaactccaggcagaacaacagcagatcccagaccctttccctggtggtcagggctcactccaacagttgccagaaaccaagctggctcttttggcctgtgtggtgccagcagaccttccatttgttgttgtgctgccagcctgcttccagagagctcagccattcttggtccaatttcagcaaaaacagcaccagcttcaacagactcaggtgttgtccaaactgcaacagcagctccatcatctgcaacccaaacttttccaatgtccagtcccactctggtgaggaagagttcttgcagttctgtcaccctctcaatgtgcctgtcagggtcaactgtgtgccttgttgcagggtagtctgcaaaagcagcagccagtgttctcacagctcttggaacatcatctctggttgccagcctcactgtgggtttgtactcagtcatggtggccctcctatagtgagtcgtattatactatgccgatatactatgccgatgattaattgtcaaaacagcgtggatggcgtctccagcttatctgacggttcactaaacgagctctgcttatatagacctcccaccgtacacgcctaccgcccatttgcgtcaatggggcggagttgttacgacattttggaaagtcccgttgatttactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcgtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgt

SEQ ID NO: 5 - protein, 300 aa, synthetic construct
  REGION 1..300: note = Description of Artificial Sequence: Synthetic polypeptide
RSDYKDDDDKGTITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSGGGGSGGGGSGEFIANPSTPSETKKPRSVAHLTGNPRSRSIPLEWEDTYGTALISGVKYKKGGLVINEAGLYFVYSKVYFRGQSCNSQPLSHKVYMRNFKYPGDLVLMEEKKLNYCTTGQIWAHSSYLGAVFNLTVADHLYVNISQLSLINFEESKTFFGLYKL

SEQ ID NO: 6 - protein, 14 aa, synthetic construct
  REGION 1..14: note = Description of Artificial Sequence: Synthetic peptide
AEAAAKEAAAKAAA

SEQ ID NO: 7 - DNA, 879 bp, synthetic construct
  misc_feature 1..879: note = Description of Artificial Sequence: Synthetic polynucleotide
  source 1..879: mol_type = other DNA
agatctgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctgaaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcaaagcatcatctcaacactaactgctgaagctgcagctaaagaagctgcagctaaagctgctgctggtaccatcaccggcacctggtacaaccagctcggctcgaccttcatcgtgaccgcgggcgccgatggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgttgacctccggcgccaccgaggccaacgcctggaagtccacgctggtcggccacgacaccttcaccaaggtgaagccgtccgccgcctcaagcggaggcggtggatcaggtggaggccatcatcaccatcaccatgaattcctcgag

SEQ ID NO: 8 - DNA, 885 bp, synthetic construct
  misc_feature 1..885: note = Description of Artificial Sequence: Synthetic polynucleotide
  source 1..885: mol_type = other DNA
agatctgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctgaaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcaaagcatcatctcaacactaactgctgaagctgcagctaaagaagctgcagctaaagctgctgctggtaccatcaccggcacctggtacaaccagctcggctcgaccttcatcgtgaccgcgggcgccgatggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgttgacctccggcgccaccgaggccaacgcctggaagtccacgctggtcggccacgacaccttcaccaaggtgaagccgtccgccgcctcaagcggaggcggtggatcaggtggaggccatcatcaccatcaccatgaattctaatagctcgag

SEQ ID NO: 9 - protein, 293 aa, synthetic construct
  REGION 1..293: note = Description of Artificial Sequence: Synthetic polypeptide
RSAPTSSSTKKTQLQLEHLLLDLQMILNGINNYKNPKLTRMLTFKFYMPKKATELKHLQCLEEELKPLKEVLNLAQSKNFHLRPRDLISNINVIVLELKGSETTFMCEYADETATIVEFLNRWITFSQSIISTLTAEAAAKEAAAKAAAGTITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSGGGGSGGGHHHHHHEFFE

SEQ ID NO: 10 - protein, 8 aa, synthetic construct
  REGION 1..8: note = Description of Artificial Sequence: Synthetic peptide
GGGSGGGT

SEQ ID NO: 11 - protein, 6 aa, synthetic construct
  REGION 1..6: note = Description of Artificial Sequence: Synthetic 6xHis tag
HHHHHH